(19) United States
(12) Patent Application Publication
(10) Pub. No.: US 2021/0147829 A1
(43) Pub. Date: May 20, 2021

(54) VARIANT NUCLEOTIDE LIBRARY

(71) Applicant: The University of Manchester, Manchester, Greater Manchester (GB)

(72) Inventor: Andrew Currin, Chadderton, Oldham, Lancashire (GB)

(21) Appl. No.: 16/621,664

(22) PCT Filed: Jun. 12, 2018
(86) PCT No.: PCT/GB2018/051591

§ 371 Date: Dec. 11, 2019

(30) Foreign Application Priority Data
Jun. 12, 2017 (GB) 1709308.9

(51) Int. Cl. C12N 15/10

(52) U.S. Cl. CPC C12N 15/1031

(57) ABSTRACT

This invention relates to a novel method for making a variant library, and to a variant library per se, preferably where the nucleic acid is DNA. The library is an OR-type library, wherein each variant of the library comprises an alternate (OR-type) mutation, using Boolean logic. These libraries, also referred to as OR-based or OR-type libraries or variant libraries comprising alternative mutations, are based upon the OR rule from Boolean logic to significantly reduce the overall size of the library, whilst still testing all the desired mutations.

This invention relates to a novel method for making a variant library, and to a variant library per se. In particular, the present invention relates to a novel method for making a variant DNA library, and to a variant DNA library per se. The present invention also provides PCR reaction mixtures comprising primers, and nucleic acid sequences, for making a variant DNA library according to the present invention. The present invention also provides a population of host cells for expression of a variant library of the present invention, preferably a DNA library.

### BACKGROUND

Protein evolution depends on the creation of diversity at the DNA level and then selection of a desired fitness at the functional protein level. Reproduction of this natural process in the laboratory is achieved through directed evolution, whereby genetic diversity is generated for a target gene (e.g. encoding a biocatalyst) in vitro, enabling isolation of variants containing a desired fitness (e.g. increased activity) through selection or screening. This process can then be repeated to iteratively improve the target molecule until an adequate fitness is achieved⁽¹⁻⁶⁾. This general outline is widely used and is a crucial technology in many applications, for example antibody discovery and optimisation, improving biocatalyst activity, and engineering biological systems in synthetic biology⁽⁷⁻⁹⁾.

Variant libraries can be generated through either random or controlled (site-directed) means. Random methods, particularly error-prone PCR⁽¹⁰⁻¹²⁾ and recombination (DNA shuffling)⁽¹³⁻¹⁶⁾ are effective in cases where little or no information is available for the target protein. In contrast, methods such as site-directed mutagenesis⁽¹⁷⁻¹⁹⁾ and gene synthesis⁽²⁰⁾ create mutations encoded by DNA oligonucleotides and hence provide means to control the type of mutations introduced and the level of diversity through the use of mixed base codons.

The relationship between a protein's sequence and function is often pictured as a 3D “landscape” and plotted on axes where the sequence (x- and y-axes) is shown versus fitness (z-axis). Proteins typically exhibit a rugged landscape, where sequences of high fitness exist around areas of low or no fitness. The strategy of directed evolution is therefore to navigate this landscape to discover the best finesses possible. Unfortunately, the size of sequence space (the total number of possible sequences) is vast and impossible to test exhaustively, even for short peptides. For example, full randomisation of a peptide of just 50 amino acids in length would produce a library of 1.13×10⁶⁵ variants.

Strategies have been developed to search for improved variants whilst at the same time making libraries of a size that can be realistically screened in the laboratory. Considerable progress has been made in the creation of reduced⁽²¹⁾ and “smart” libraries⁽²²⁻²⁴⁾. These strategies reduce the level of diversity and the redundancy of the mixed base codons used, generally for a small and select number of codons. However they do not address the need to mutate a greater number of codons, as even these strategies create libraries too large to test experimentally when mutating large numbers of codons simultaneously.

The present invention aims to overcome and/or ameliorate problems associated with DNA libraries of the prior art.

### BRIEF SUMMARY OF THE DISCLOSURE

In a first aspect, there is provided a variant nucleic acid library of a nucleic acid molecule of interest, comprising a population of variant nucleic acid molecules wherein each variant comprises an alternative mutation in a first target region to other members of the population. In an embodiment, each variant may further comprise an alternate (OR-type) mutation in a second or further target region, to other members in the population. Consequently, mutations may be shared by some (but not all) of the other members of the library. A variant nucleic acid may comprise an OR-mutation simultaneously in two or more target regions. This may be referred to as a combinatorial library. Alternatively, a library may comprise two or more variants of a nucleic acid molecule of interest, wherein each variant comprises an OR-type mutation in a single target region. In a first aspect, a variant library may comprise a population of polymers which are not nucleic acids, wherein each variant comprises an alternative mutation in a first target region to other members of the population. Polymers may include polypeptides, polysaccharides etc.

In a second aspect, there is provided a method of making a variant library of a nucleic acid molecule of interest, wherein the library comprises a population of variant nucleic acid molecules, wherein each variant comprises an alternative (Boolean OR-type) mutation in at least one target region, the method comprising: amplification of the nucleic acid molecule using a population of mutagenic primers encoding the desired alternative (Boolean OR-type) mutations. Therefore, a variant nucleic acid library according to the first aspect can be amplified from an existing nucleic acid molecule or assembled from overlapping nucleic acid molecules (for example by gene synthesis), such that the variant nucleic acid library produced contains alternative OR-type mutations in one or more target regions.

In an embodiment of the second aspect, there is provided a method of making a variant library of a nucleic acid molecule of interest, wherein the library comprises a population of variant nucleic acid molecules, wherein each variant comprises an alternative (Boolean OR-type) mutation in a first target region, the method comprising:
I) incubating i) a nucleic acid molecule of interest; ii) a limited concentration of two or more mutagenic primers which each hybridise to a first strand of the nucleic acid molecule; and iii) an excess concentration of a first primer which hybridises to a second strand of the nucleic acid molecule;
II) maintaining the incubation under suitable conditions for X number of rounds of amplification, wherein X is n+y where n is the number of PCR rounds required to deplete the one or more mutagenic primers, and y is 2 or more; to generate two or more single stranded amplification products wherein each two or more single stranded amplification product comprises an alternative (OR-type) mutation in the first target region; and
III) incubating i) the two or more said single stranded amplification products of the first target region which hybridise to the second strand of the nucleic acid molecule of interest; and ii) a second primer which hybridises to the first strand of the nucleic acid molecule of interest to provide two or more double stranded amplification products;
IV) maintaining the incubation under suitable conditions for sufficient rounds of amplification to generate the two or more double stranded amplification products wherein each double stranded amplification product comprises an alternative (OR-type) mutation.

In an embodiment of the second aspect, the method comprises a method of making a variant library of a nucleic acid molecule, wherein each variant of the library further comprises an alternative (OR-type) mutation in a second or further target region, the method further comprising:
in step I) incubating i) a limited concentration of two or more second or further mutagenic primers which each hybridise to a first strand of the nucleic acid molecule for amplification of a second or further target region; and ii) an excess concentration of a first primer which hybridises to a second strand of the nucleic acid molecule;
in step II) maintaining the incubation under suitable conditions for X number of rounds of amplification, wherein X is n+y where n is the number of amplification rounds required to deplete the mutagenic primers, and y is 2 or more; to generate two or more single stranded amplification products of a second or further target region, each two or more single stranded amplification product comprises an alternative (OR-type) mutation in the first target region and an alternative (OR-type) mutation in the second or further target region; and
in step III) incubating i) the two or more said single stranded amplification products of the second or further target region which hybridise to the second strand of the nucleic acid molecule of interest; and ii) a second primer which hybridises to the first strand of the nucleic acid molecule of interest to provide two or more double stranded amplification products of the second or further target region;
in step IV), maintaining the incubation under suitable conditions for sufficient rounds of amplification to generate the two or more double stranded amplification products of the second target region wherein each double stranded amplification product comprises an alternative (OR-type) mutation in the second or further target region.

Therefore, in an embodiment there is provided a method of making a variant library of a nucleic acid molecule of interest, wherein the library comprises a population of variant nucleic acid molecules, wherein each variant comprises an alternative (Boolean OR-type) mutation in at least one target region, the method comprising:
I) incubating i) a nucleic acid molecule of interest; ii) a limited concentration of two or more first mutagenic primers which each hybridise to a first strand of the nucleic acid molecule for amplification of a first target region and optionally a limited concentration of two or more second or further mutagenic primers which each hybridise to a first strand of the nucleic acid molecule for amplification of a second or further target region; and iii) excess concentration of a first primer which hybridises to a second strand of the nucleic acid molecule;
II) maintaining the incubation under suitable conditions for X number of rounds of amplification, wherein X is n+y where n is the number of amplification rounds required to deplete the mutagenic primers and y is 2 or more; to generate two or more single stranded amplification products of said first and optionally second or further target region, each two or more single stranded amplification product comprises an alternative (OR-type) mutation in the first target region and optionally an alternative OR-type mutation in the second or further target region; and
III) incubating i) the two or more said single stranded amplification products which hybridise to the second strand of the nucleic acid molecule of interest; and ii) a second primer which hybridises to the first strand of the nucleic acid molecule of interest to provide two or more double stranded amplification products comprising an alternative (OR-type) mutation in a first and optionally second or further target region;
IV) maintaining the incubation under suitable conditions for sufficient rounds of amplification to generate the two or more double stranded amplification products wherein each double stranded amplification product comprises an alternative (OR-type) mutation in a first and optionally second or further target region.

The single stranded amplification products may comprise a single target region, or multiple (first, second or further target regions). Separate single stranded amplification products may be generated for first, second or further target regions. Each target region comprises an OR type mutation.

In a third aspect of the invention, there is provided an incubation comprising i) a limited concentration of two or more mutagenic primers which each hybridise to a first strand of the nucleic acid molecule; ii) a first primer which hybridises to a second strand of the nucleic acid molecule; and iii) nucleic acid molecule of interest; and optionally iv) said two or more single stranded amplification products and/or said two or more double stranded amplification products.

An incubation of the third aspect may alternatively or additionally comprise i) a second primer which hybridises to the nucleic acid molecule; optionally in combination with one or more of i) to iv) of the third aspect.

In a fourth aspect of the present invention there is provided a population of variant host cells, each variant comprising one or more variant nucleic acid molecules of a variant nucleic acid library, wherein each variant nucleic acid molecule comprises an alternative mutation in a first target region to other members of the population. In an embodiment, each variant may further comprise an alternative (OR-type) mutation in a second or further target region. The variant nucleic acid may comprise an OR-type mutation simultaneously in two or more target regions or each variant may comprise an OR type mutation in a single target region. According to the fourth aspect, the library may be a population of variant host cells, each variant comprising one or more variant polymer (not nucleic acid) molecules of a variant polymer library, wherein each variant polymer molecule comprises an alternative mutation in a first target region to other members of the population.

### BRIEF DESCRIPTION OF THE DRAWINGS

Embodiments of the invention are further described hereinafter with reference to the accompanying drawings, in which:

FIG. 1 shows a summary of the procedure for site-directed mutagenesis using asymmetric PCR. Arrows indicate the DNA oligonucleotides used as PCR primers (arrow head signifying 3′ end). A) A mutagenic primer at a low concentration (mutagenic primer) is used together with a primer of 20× concentration (excess primer). The PCR reaction therefore generates a ssDNA product (containing the mutations encoded by the primer). B) This product is then used as a megaprimer to generate the full-length gene in the second PCR, the products of which encode all the desired mutations.

FIG. 2 illustrates the difference between products of symmetric and asymmetric PCR amplification when analysed by capillary electrophoresis.

FIG. 3 Comparison of OR-type and conventional libraries derived by site-directed mutagenesis. When a single target region is to be mutated (a “set”, highlighted in red), all codons are mutated simultaneously using existing AND-type mutations (A.). Single set OR-type libraries can include one codon mutation per region (codon or codons, B.) or multiple codons per region (C.), consequently members of each library encode mutations in region 1 or 2, but not both. For combinatorial libraries with multiple sets (highlighted in red and green, D.), this can be extended to create OR-type mutations at multiple positions, thus creating AND-OR libraries with single (E.) or multiple (F.) mutations per region.

FIG. 4 shows the creation of OR-type and combinatorial OR-type libraries. A. A set of multiple mutagenic primers, encoding mutations at region 1 or region 2, are used in the same PCR to create mutagenic megaprimers. Synthesis of the full-length sequence using these megaprimers generates an OR-type library, containing DNA strands with mutations at either region 1 or region 2. B. Additional sets of mutations (regions 3 and 4) can be used to create a second set of mutagenic megaprimers, which when pooled with set 1 in the second PCR generates combinatorial OR-type mutations (AND-OR libraries).

FIG. 5 shows a sequencing alignment of an NNK library generated using asymmetric PCR. The codon for aspartic acid (GAT) was mutated with the NNK mixed base codon. Sequencing data (from single colonies selected from this library) show the correct representation of each of the mixed bases (N=A, T, G, C and K=G, T).

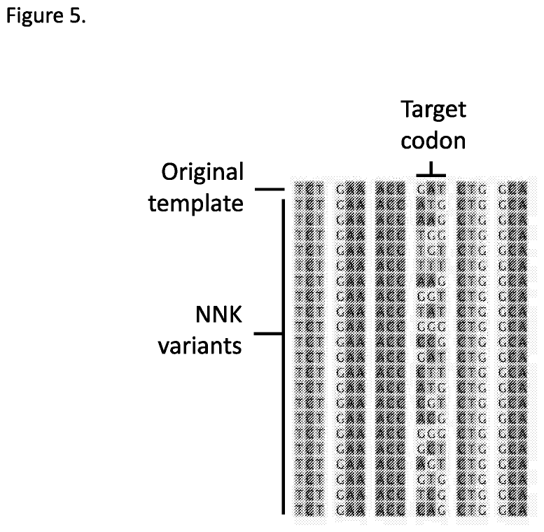

FIG. 6 shows a schematic for asymmetric PCR to generate OR-based libraries. A) Multiple oligonucleotides can be used as mutagenic primers in the same PCR, hence the resulting gene library encodes a mixture of mutations from each of the mutagenic oligonucleotides, but not multiple mutations from different oligonucleotides. B) Sequencing data from selected colonies of OR-based libraries. Two target regions (containing 3 and 2 codons, respectively) were mutated simultaneously to produce OR-based libraries, encoding mutations in region 1 OR 2.

FIG. 7 shows sequencing data from selected colonies of OR-type libraries. A. Two target regions (containing 1 amino acid each) were mutated using the NNK codon (N=A, T, G, C and K=G, T). B. Two target regions (target 1=3 codons, target 2=2 codons) were mutated using our mixed base codon design (table 1).

FIG. 8 shows the utilisation of the OR rule for creation of variant libraries using MAO-N (Monoamine oxidase-N, A. niger). A) Every residue mutated is highlighted (blue) on the MAO-N crystal structure (PDB code 2VVM), a total of 54% of the total MAO-N protein. B) An example of the MAO-N mutagenesis. Three different positions (starting residues 127,138 and 339) of MAO-N were targeted, each position contained multiple mutagenic primers. For each sample the asymmetric PCR control (A) and full length (FL) library is shown. C) An example of the colony-based colourimetric screening used to test for catalytic activity.

FIG. 9 shows the design of OR-type libraries and their efficiency to screen multiple amino acid mutations. A. Following selection of a target amino acid sequence (here, YSERIDQIRDE, “WT amino acid”) and it's coding sequence (“WT DNA”), mutagenesis regions were selected (shown as [1]-[4]). B. Visualisation of the OR-type mutations on the MAO-N structure (PDB code 2VVM). C. The OR-type library produced by this approach encodes 5136 genetic combinations, a 1.1×10⁸-fold reduction compared to simultaneous AND-type mutations.

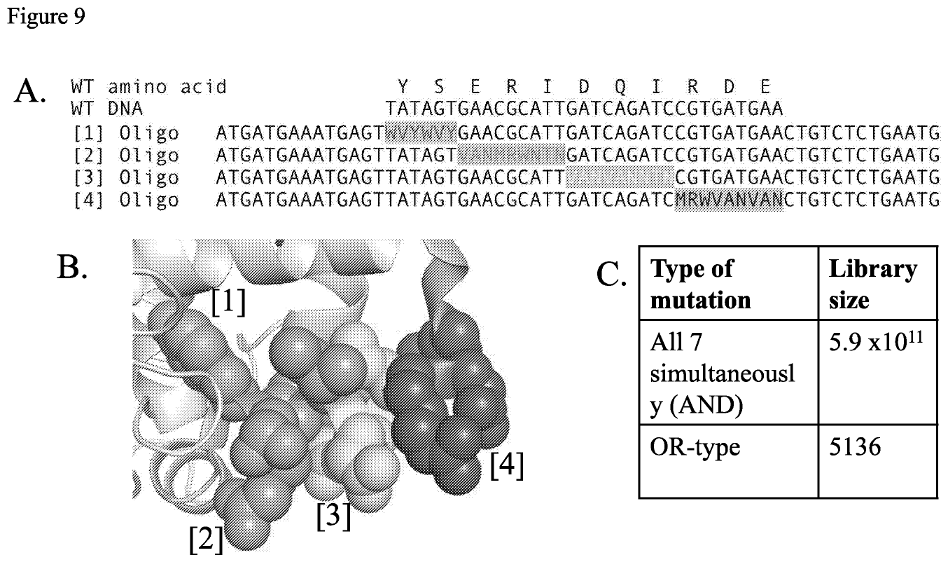

FIG. 10 is a schematic to show the use of the present invention to create combinatorial OR-based libraries. Multiple positions in a target sequence can be selected and used to create complementary megaprimers, which can be combined to produce OR type mutation at multiple positions.

FIG. 11 shows the creation of combinatorial libraries for MAO-N. Two regions were selected for combinatorial mutagenesis due to their close proximity to the substrate binding pocket of MAO-N (Atkin et al: J Mol Biol 2008; 384:1218-1231; and Rowles et al; ChemCatChem. 2012; 4 (9): 1259-1261 DOI: 10.1002/cctc.201200202). A) Synthesis of the two megaprimers using asymmetric PCR (“A”) and assembly into a single MAO-N library (“FL”). B) Alignment of the sequencing data from E. coli colonies of this library, showing that different combinations of mutations at each region were been created.

FIG. 12 shows the improvement in activity for MAO-N variants. A. The most significant improved activity to the primary target non-native substrate α-methylbenzylamine was demonstrated by the D5 variant A289V, exhibiting a 1.6-fold increase to that of D5 and a 1210-fold increase to that of the wild type. B. Improved activity to three native amine substrates was shown by the D5 variant F128L, with a k_{cat} between 1.6 to 2.25-fold higher than the WT, and 2.2 and 3-fold higher than the parent D5 variant.

FIG. 13 Structural analysis of selected mutations and in vitro amino acid selection (using MAO-N D5 structure, PDB code 2VVM). A. Highlighting 6 mutations that confer an increase in k_{cat} for MAO-N (including N336S) shows that they are distributed throughout the protein structure and mainly not within the active site, which is indicated by an L-proline ligand and FAD cofactor. B. The amino acid changes A266V and C50T that confer activity to the novel substrate CHA are located distal to the active site; their distances to the amine of the FAD cofactor are shown. C. Every amino acid mutated in this study is shown, with its colour denoting whether it i) showed strong selection for the wild-type amino acid (white); ii) exhibited robustness, where at least one alternative mutation could be accommodated whilst still maintaining activity (grey); and (iii) exhibited strong selection for a new mutation that increased k_{cat} (black). Images generated using PyMol.

FIG. 14. Combinatorial OR-type libraries (AND-OR libraries) for CASTing. A. Sets 1 (residues [209]-[213]) and 2 ([241]-[245]) each contained 5 amino acids for mutagenesis. The AND-OR library created every mutation combination between sets 1 and 2, i.e. {[1] AND [1]} OR {[1] AND [2]}, etc. B. The “hit” combination, exhibiting novel activity to non-native substrates, encoded mutations at the [1] (A2095) and [5] (L245C) positions. C. Simultaneous mutagenesis using the NNK codon using conventional AND-type mutations produces over 10¹⁵ genetic combinations, while the corresponding combinatorial OR-type library encoded 25600 combinations, a 4.4×10¹⁰-fold reduction in library size.

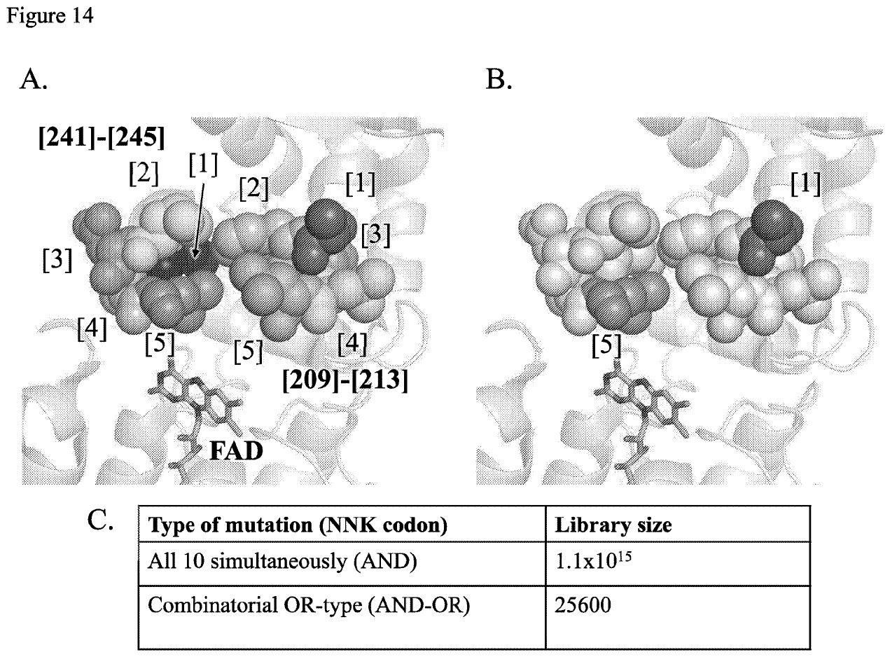

### DETAILED DESCRIPTION

The present invention relates to an OR-type library, wherein each variant of the library comprises an alternate (OR-type) mutation, using Boolean logic. These libraries, which herein are referred to as OR-based or OR-type libraries or variant libraries comprising alternative mutations, are based upon the OR rule from Boolean logic to significantly reduce the overall size of the library, whilst still testing all the desired mutations. This enables the mutation of multiple amino acids in a single sample without testing all residues simultaneously with each other. The effect of these mutations is therefore additive (the OR rule in Boolean logic), a feature that reduces the overall library size dramatically, often by several orders of magnitude. Consequently, this provides the opportunity to mutate a far greater number of positions in a target sequence, greatly facilitating the search for “hits” from an otherwise vast sequence space. Therefore, the variants in the library each comprise a different mutation, or set of mutations, in any one target region, but do not comprise all combinations of mutations in any one target region. This may be referred to herein as “alternative” mutations between the variants in a library, or OR-type mutations. A library of the present invention is achieved by employing multiple mutagenic DNA primers in the same amplification reaction. For example, when using two mutagenic primers, each DNA strand produced will encode a mutation from one primer OR the other primer, but will not comprise mutations of both primers. This is referred to herein as alternative or OR-type mutations. Consequently, OR-based libraries can be used to mutate a greater number of codons without creating a large library size. The present invention relates to methods for producing such a library, for example using an asymmetric amplification reaction.

### Definitions

A nucleic acid molecule of interest is any nucleic acid molecule of which it may be desired to identify variants, typically variants having an improved functionality.

A variant nucleic acid molecule is a nucleic acid molecule of interest which comprises one or more mutations in the nucleic acid sequence, different from the starting sequence of the nucleic acid molecule of interest.

A library is a collection of variant molecules. These may be polymers, including polypeptides, polysaccharides and nucleic acid molecules. They may be either heterogeneous nucleic acid sequences or related nucleic acid sequences such as variants of a single sequence.

A variant DNA library is a population of variant nucleic acid molecules. A variant DNA library may be a directed or specifically engineered library (as opposed to a randomly generated library).

A population of variant polymeric molecules of interest is a mixture of variants of a polymeric molecule of interest. The polymeric molecule may be a nucleic acid, polypeptide or polysaccharide, for example. A population is preferably a mixture of variants of a single polymeric molecule of interest.

A mutation is a modification of the starting sequence of a polymeric molecule. In a polypeptide, a mutation may be a modification of the amino acid sequence for example by deletion, addition or substitution of one or more amino acids of the starting sequence. In a nucleic acid, a mutation is a modification of the starting sequence of a nucleic acid molecule of interest, for example by deletion, addition or substitution of one or more nucleotides of the starting sequence.

An alternative, alternate, OR-based or OR-type mutation is a mutation at a defined position which is not shared by other variants of the polymeric molecule of interest in the population or library. The same mutation may be shared by a subset of other variants within the population or library, but not all variants in the population or library.

The term complementary refers to a nucleic acid sequence of bases that can form a double-stranded nucleic acid structure by matching base.

A primer is an oligonucleotide which binds to a strand of a nucleic acid molecule of interest and serves as a starting point for polymerisation (replication) of the nucleic acid molecule of interest, typically along a complementary strand of the nucleic acid molecule under suitable conditions, thus replicating the complementary strand in the new strand.

A primer pair is two primers which in combination serve as starting points for replication or amplification of a nucleic acid molecule of interest.

A mutagenic primer is a primer which is capable of introducing a mutation into an amplification product of a nucleic acid molecule of interest.

A megaprimer is a long primer (typically greater than 60 nucleotides). Herein, a megaprimer may be produced in a PCR and comprises the mutation to be introduced into the amplified product. Herein, a first amplification product may serve as a megaprimer.

An amplification product is a nucleic acid molecule generated in an amplification reaction (for example, by PCR), by synthesis of a new nucleic acid molecule from a primer sequence.

A target region is a partial sequence of the nucleic acid molecule of interest, which is subject to variation to produce a library of the present invention.

A defined position is a specific place in the polymeric molecule of interest that is to be mutated.

An amplification reaction refers to any process for repeatedly replicating a nucleic acid to generate multiple copies. PCR refers to the polymerase chain reaction, and means a method for amplifying a nucleic acid sequence.

Asymmetric amplification is a method where one strand of a nucleic acid molecule is amplified in preference to the other strand. Asymmetric PCR uses PCR as the amplification reaction, and preferential amplification of one strand occurs by use of an unequal concentration of primers.

An amplification product is a copy or replicated sequence of the nucleic acid molecule of interest, or a portion thereof (for example a target region). An amplification product may comprise the mutations encoded by the mutagenic primers.

A polymeric molecule may be (but is not limited to) a nucleic acid molecule, a polypeptide or a polysaccharide.

Throughout the description and claims of this specification, the words “comprise” and “contain” and variations of them mean “including but not limited to”, and they are not intended to (and do not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

### General

A nucleic acid molecule of interest may be any molecule that can be used to create a library. This may be, but is not limited to, DNA (including genomic DNA), RNA (including mRNA), and/or complementary DNA (cDNA). Also included are other polymers made up of simpler units joined in a chain (including polysaccharides). The nucleic acid molecule may be single or double stranded. It may be linear or circular (e.g. plasmid), in fragments (e.g. overlapping oligonucleotides) or in a single molecule, synthetic or from a native source. It may be cosmid DNA, bacterial artificial chromosome (BAC), or yeast artificial chromosome (YAC). In some aspects, the nucleic acid molecule is generated from chemical synthesis, reverse transcription, DNA replication or a combination of these generating methods. A nucleic acid molecule of interest may code for a polypeptide or may be a non-coding sequence, for example a regulatory sequence such as a promoter or enhancer sequence. The sequence of a nucleic acid molecule of interest may be a native sequence or a non-native sequence. The sequence of the nucleic acid molecule of interest prior to application of a method of the present invention may be referred to herein as the starting sequence of the nucleic acid molecule of interest.

A nucleic acid molecule of interest may be synthetic, or may be derived from any source, including for example organelles, cells, tissues, organs, organisms or viruses. A nucleic acid molecule of interest could also be created de novo (e.g. by gene synthesis from overlapping oligonucleotides) during the method. A cell may be derived from any prokaryotic, archaeal or eukaryotic source. A cell may include, without limitation, bacterial cells, fungal cells, plant cells (including vegetable cells), protozoan cells, and animal cells. Such animal cells include, but are not limited to, insect cells, nematode cells, avian cells, fish cells, amphibian cells, reptilian cells, and mammalian cells. In some aspects, the mammalian cells include human cells.

Methods for obtaining a nucleic acid molecule of interest as the starting point for a method of the present invention are known in the art, including those described by Maniatis et al., Molecular Cloning: A Laboratory Manual, Cold Spring Harbor, N.Y., pp. 280-281 (1982). Nucleic acid molecules obtained from biological samples may be fragmented to produce suitable fragments for analysis.

A nucleic acid molecule of interest may be any suitable size, which can be accommodated in an amplification reaction such as PCR. Where a gene or sequence of interest is too long (for example above 6000 bp), it may be fragmented using any suitable method into shorter fragments for use in the present invention. Suitable sizes for amplification will be known to persons skilled in the art. A method of the invention may comprise the step of dividing a gene or sequence to provide one or more shorter fragments as the nucleic acid molecule of interest. A shorter fragment of a full length sequence of interest could be amplified in the reaction.

A nucleic acid molecule of interest may be any nucleic acid that can be used to encode a functional molecule. A functional nucleic acid molecule may include, but is not limited to, DNA coding sequences (e.g. encoding a binding protein such as an antibody, a catalyst, or regulatory protein such as a transcription factor), non-coding sequence (e.g. a ribosome binding site, a promoter, enhancer or other regulatory sequence) or other functional molecule (e.g. ribozyme or riboswitch), or a fragment or combined assembly (e.g. ribosome binding site with coding sequence) thereof.

The starting sequence of a nucleic acid molecule of interest is understood to mean a nucleic acid sequence which can be native (wild-type sequence), but it does not necessarily have to be native. The method according to the invention can also start from multiple different DNA starting sequences. Preferably, the method according to the invention starts from an individual DNA starting sequence.

A target region may be a coding or non-coding sequence, or a combination thereof. In the present invention, which reduces library size by providing OR-based mutations, an amplification product of a single target region will comprise a mutation from one or more mutagenic primers. Two or more target regions may be provided in a nucleic acid sequence of interest. The division is preferably carried out purely notionally, i.e., preferably no cleavage of the DNA starting sequence takes place (e.g., with restriction endonucleases). A target region may independently comprise at least 20, 30, 40, 50, 60, 70, 80, 90, 100, 120, 150, 180, 200, 500 or 1000 nucleotides, or any integer or range in-between, such that it is able to act as a primer in the amplification reaction. A suitable length can be determined by a person skilled in the art, based upon the reaction parameters. By a further target region means a third, fourth, fifth, sixth or more target region or regions. A nucleic acid molecule of interest may be notionally divided into any number of target regions, which may depend upon the size of the target molecule of interest and the number of mutations to be made.

A mutation may be in a coding or non-coding sequence. When in a coding sequence, a mutation in the DNA sequence may introduce a mutation at protein level. A mutation may be a substitution, addition or deletion of a nucleotide in the starting sequence of a nucleic acid molecule of interest. A mutation may be a substitution, addition or deletion of one, two or three or more adjacent nucleotide residues in the starting nucleic acid sequence. When in a coding sequence, a mutation may be in a multiple of three nucleotide residues, thereby introducing corresponding changes at the protein level. A mutation may be the substitution, addition or deletion of three or more, for example six, none, twelve, or more adjacent nucleotide residues. Also included are mutations spaced separately or both adjacent and separately.

A mutation comprising a substitution, addition or deletion mutation may relate to an individual nucleotide residue or up to three nucleotide residues immediately following one another within the starting sequence, but not to multiple nucleotides which are separated from each other within the starting sequence by one or more nucleotide residues. Where two substitutions, additions or deletions do not form part of the same codon (in a coding sequence), they are separate mutations. If, in a coding sequence, the substitution, addition or deletion is separated by one or more nucleotides from another such mutation, but the two mutations affect the same codon, they are a single mutation for the purposes of the present invention.

The position of a mutation is the nucleotide position(s) at which the mutation is effective. In the case of substitutions, the mutation position can be specified by naming the nucleotide position in the starting sequence. In the case of insertions, the mutation position can be specified by naming the two nucleotide positions between which the insertion lies. In the case of deletions, the mutation position can be specified by naming the deleted nucleotide.

Where two or more target regions are mutated in a target nucleic acid molecule of interest, using the method of the invention, the two or more target regions are mutated such that a variant of the library will comprise a mutation in two or more target regions in random combinations, such that each variant has a unique combination of mutations (i.e. a unique combination of mutations in the first, second or further target regions, such that the combination in the first, second or further target region is not shared by another variant). Therefore, each mutation in each target region is an OR-type mutation.

Substitution, insertion, and deletion of nucleotide residues in a nucleic acid sequence can be clearly identified by comparison of the starting nucleic acid sequence with the sequence of the amplification product, with the non-mutant starting sequence serving as the reference.

A primer for use in the present invention is an oligonucleotide suitable for use in an extension reaction, such as an amplification reaction, such as PCR. A primer will, by definition, be capable of initiating polymerisation of the target nucleic acid molecule. A primer may be RNA or DNA or cDNA. Preferably, it will be the same as the nucleic acid molecule of interest to be amplified. Therefore, where the nucleic acid molecule is DNA, the primer will be DNA; where the nucleic acid molecule of interest if RNA, the primer will be RNA and so on. It may be synthetic or naturally occurring. It may be single stranded, comprising a sequence that is capable of hybridizing with a sequence of the nucleic acid molecule of interest. A primer may be any suitable length, defined by factors such as the length of the target sequence, GC content and temperature at which the primer will hybridise (Tₘ). A primer is generally of sufficient length to hybridise to a nucleic acid sequence, and enable polymerisation thereof to provide an amplification product. A primer may still hybridise to a target even though its sequences are not fully complementary to the target. Therefore, a primer suitable for use in the present invention may be between, but not limited to, 20 and 200 base pairs in length.

The region of the nucleic acid molecule of interest to which a primer binds may be referred to herein as the primer binding site.

A primer may be a mutagenic primer, meaning that it will comprise a sequence which introduces a mutation into the starting sequence of the target molecule of interest. Suitably, it is capable of introducing a mutation into a target region of a nucleic acid molecule of interest. By “introducing a mutation”, it is meant that an amplification product of the starting sequence comprises a mutation corresponding to the mutation present in the primer. Therefore, a mutagenic primer for hybridisation to the first strand of a nucleic acid molecule of interest will comprise a sequence which varies from the exact complement of said first strand. A mutagenic primer may comprise a single mutation or a set of multiple mutations. Each mutation may independently be substitution, addition or deletion of a nucleotide residue. A mutagenic primer may comprise the substitution, addition or deletion of 1, 2, 3, or more nucleotides up to n, where n is the length of the mutagenic primer. A mutation may be a single point mutation. Multiple mutations may be adjacent or may be separated by one or more non-mutant residues. The mutagenic primer may vary by substitution, addition or deletion of one, two or three or more adjacent nucleotide residues, such that it introduces a mutation of two or more adjacent nucleotides. When in a coding sequence, a mutagenic primer may introduce a mutation within a codon, thereby introducing corresponding changes at the protein level. The sequence of a mutagenic primer can be either specifically defined (using the nucleotides A, T, G or C) or not specifically defined (for example, using the IUPAC code for mixed base nucleotides). The type, number or position of mutations in a mutagenic primer is not limited in the design or production of an OR-type library of the invention, neither is the number of mutagenic primers to be used. In the creation of OR-type libraries, multiple mutagenic primers can be used, each one encoding a different set of mutations.

In the present invention, the mutagenic primers in an amplification reaction are each provided in a limited concentration. This means that in an amplification reaction of X rounds, the mutagenic primers will become depleted. The depletion ensures that each of the mutations of the mutagenic primers are replicated in an amplification product. The mutagenic primers are preferably provided in a concentration such that they become depleted in n number of amplification rounds. Herein, n may be 2, 3, 4, 5, 10 or more, up to the total number of cycles used in the amplification. Consequently, when n is reached the first primer continues to amplify one strand. The total number of amplification cycles for any reaction may be determined by a person skilled in the art.

A limited concentration of a mutagenic primer may be defined in relation to the concentration of the first or second primer which is provided in a 5, 10, 15, 20, or 25-fold molar excess. For example, a mutagenic primer may be provided at a concentration of 25 nM and a first primer at a concentration of 500 nM. The ratios of mutagenic primer:first primer and mutagenic primer:second primer (and so on) may be the same or may be different, such that they yield the desired amplification product.

The total number of mutagenic primers used in a method of the invention will depend upon the size of the nucleic acid molecule starting sequence or target region, the number of target regions, and the number of mutation sites to be introduced. The number of variants generated will depend on the number of mutagenic primers used, the number of target regions mutated and the number of mutation sites to be introduced. Therefore, the number of mutagenic primers to be used in a method of the invention can be determined by a person skilled in the art, but may be for example between 2 and 10000000.

A mutagenic primer may bind to bases including the 5′ end, 3′ end or any intervening sequence of a starting sequence or target region.

A primer binding site for a mutagenic primer will enable hybridisation and subsequent replication of a target region when the primer binds thereto under conditions suitable for amplification. A primer binding site for a mutagenic primer may be wholly within, partially within or near to a target region. A first mutagenic primer will therefore hybridise to a sequence which enables amplification of a first target region. Similarly, a second or further mutagenic primer will therefore bind to a sequence which enables amplification of a second or further target region, respectively.

A first primer used in the present invention serves to amplify the second strand of the nucleic acid molecule of interest. A first primer is provided in excess concentration (i.e. 5, 10, 15, 20 or 25 fold or more), compared to mutagenic primers, suitably 20 fold more than the cumulative mutagenic primers present. The excess concentration is sufficient to enable X rounds of amplification, wherein X is n+y, where n is the number of rounds in which the two or more first and/or second mutagenic primers become depleted (e.g. 1, 2, 3, 4, 5, 10 or more); where y, which is the number of rounds of PCR after depletion of the mutagenic primers, which may be 2, 3, 4, 5, 10 or more. The excess concentration of a first primer compared to the mutagenic primers means that two or more single stranded amplification products of a target region, each comprising an alternative (OR-type) mutation, are generated.

The first primer may be mutagenic, but preferably is non-mutagenic which means that its sequence is sufficiently complementary to the primer binding site of the second strand of the nucleic molecule of interest such that it can hybridise to the second strand and initiate polymerisation (the second amplification product). If primers are non-mutagenic, the first primer may therefore have a sequence which is at least 95%, 97%, 98%, 99%, 99.5% or most preferably 100% complementary to the sequence of the primer binding site on the second strand of the nucleic acid molecule of interest.

A second primer serves, in combination with the single stranded amplification product acting as a megaprimer, to amplify the nucleic acid molecule to provide two or more double stranded amplification products of the target region, wherein each two or more double stranded amplification products of the target region comprise an alternative, OR-type mutation. The second primer may be mutagenic, but preferably is non-mutagenic which means that its sequence is sufficiently complementary to the primer binding site of the second amplification product such that it can hybridise to the second amplification product and initiate polymerisation (producing the double stranded amplification product). The second primer may therefore have a sequence which is at least 97%, 98%, 99%, 99.5% or most preferably 100% complementary to the sequence of the primer binding site on the second amplification product.

A primer binding site for a first or second primer may be provided at any suitable site on either first or second strand of the nucleic acid molecule of interest or the first amplification product, respectively. Preferably, a primer binding site is provided partially or wholly within a target region, or flanking a target region. By flanking is meant that the primer binding site lies immediately or approximately adjacent to the 3′ or 5′ end of a target region. By approximately adjacent means that it may be distanced from a 3′ or 5′ end of the target region, for instance by 1, 2, 3, 4, 5, 10, 15, 20, 25, or 30 or more nucleotides.

The starting nucleic acid molecule of interest, or a previous amplification product, may serve as a template for replication.

A two or more single stranded amplification product is a single stranded nucleic acid molecule which each comprise a mutation of a first mutagenic primer. Each two or more single stranded amplification products do not comprise the same mutation. Therefore, the mutations provided in two or more single stranded amplification products are alternative, “OR” type mutations. The two or more single stranded amplification products of a first, second and/or further target region serve as primers (also referred to as megaprimers) for a second PCR reaction, in combination with a second primer, to amplify the nucleic acid molecule. The presence of the mutations in the two or more single stranded amplification products (megaprimers) means that the second PCR reaction results in two or more double stranded amplification products wherein each amplification product comprises an alternative (OR-type) mutation in each of the first, second and/or further target regions. Preferably, the two or more single stranded amplification products and the second primer are provided in approximately equal concentrations, in order to generate a double stranded amplification product. Therefore a method of the invention may comprise the step of isolating the two or more single stranded amplification products, prior to the second amplification reaction. The method may comprise determining the concentration of the two or more single stranded amplification products, and using the value to determine the amount of second primer for use in the second reaction.

Reference to “two or more” amplification products means two or more variants of an amplification product. The number of variants of an amplification product will preferably reflect the number of variants encoded by the mutagenic primers used in the first amplification reaction. Each variant amplification product may be present in multiple copies, for example after n rounds of amplification a first amplification product may be present in thousands of copies. The abundance of each amplification product relates to the number of mutations made by the mutagenic primer. For example, if the size of the library is 2, then each product will have an abundance of 50%. For a library of size 100, each amplification product will have an abundance of 1%. In the first amplification reaction, n may be calculated in order to provide a pre-determined amount of the two or more single stranded amplification products which serves as a primer for the second amplification reaction. Y may be calculated depending upon the estimation of the single stranded amplification product present after n rounds of amplification.

Any suitable amplification reaction may be used in the present invention. Such amplification reactions include, without limitation, PCR, gene synthesis (overlap extension PCR), loop mediated isothermal reaction, nucleic acid sequence based amplification, strand displacement amplification, rolling circle amplification, multiple displacement amplification, and ligase chain reaction. PCR is an in vitro amplification procedure based on repeated cycles of denaturation, oligonucleotide primer hybridisation, and primer extension by template dependent polynucleotide polymerase, resulting in the exponential increase in copies of the desired sequence of the polynucleotide analyte flanked by the primers. The two different PCR primers, which anneal to opposite strands of the DNA, are positioned so that the polymerase catalyzed extension product of one primer can serve as a template strand for the other, leading to the accumulation of a discrete double stranded fragment whose length is defined by the distance between the 5′ ends of the oligonucleotide primers. The primers flank and attach to the sequence to be amplified. Methods for PCR are well known in the art^{(17, 27)}.

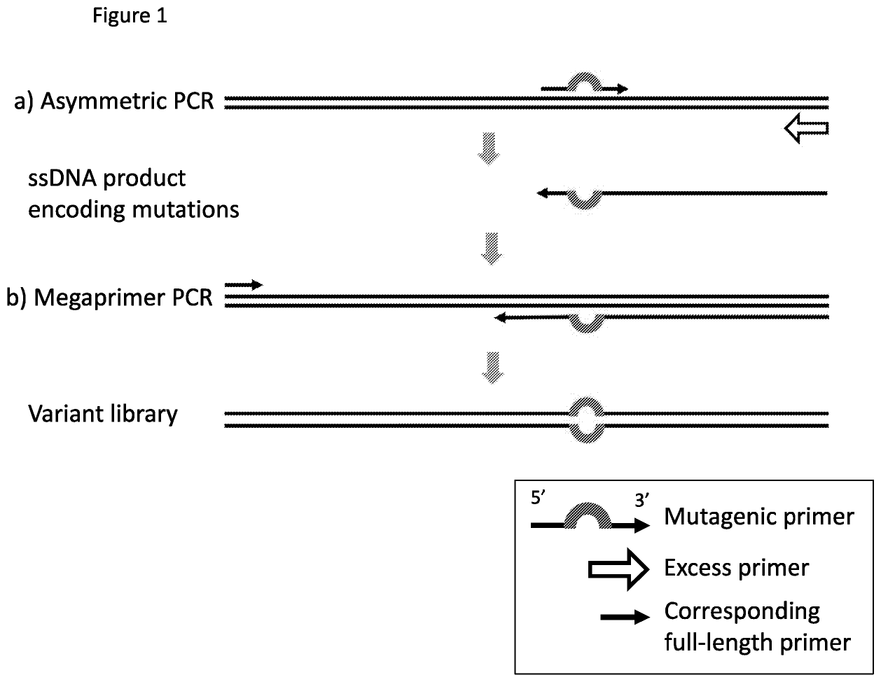

Herein, asymmetric amplification is utilised to introduce a mutation into a nucleic acid molecule of interest. Numerous studies have previously utilised asymmetric PCR for the purposes of site-directed mutagenesis^{(25,27-29,26)} of a nucleic acid molecule of interest. In the process, the first step consists of an asymmetric amplification reaction to generate a single stranded nucleic acid product (herein referred to as the first amplification product), which is created using an unequal concentration of primers. A low (limiting) concentration of one or more mutagenic primers become depleted during the early cycles of the amplification reaction, thereby ensuring that all mutagenic primers are used and each of the mutations encoded by a mutagenic primer is replicated in a first amplification product. The high concentration (excess) primer amplifies the second strand of the DNA product, to produce a second amplification product, complementary to the first amplification product. This second amplification product is a single stranded nucleic acid molecule comprising a mutation encoded by a one or more mutagenic primer. This product is then used as a megaprimer in a second amplification reaction to amplify the full-length gene sequence library (FIG. 1). The method according to the invention is suitable for producing a variant library of DNA sequences. These DNA sequences can then be translated into a variant library of protein molecules in which targeted mutations are encoded at defined amino acid positions.

The method according to the invention is preferably carried out in a targeted manner, i.e., proceeds in a directed manner, in a rational manner, or predetermined to a certain degree, at least in part, but not arbitrarily or randomly (randomized). For a coding sequence, the codon for any amino acid may be randomised (i.e. to all other possible amino acids) but this randomisation is targeted to that codon, and encoded by the oligonucleotide. Variants may be created by mixtures of bases (e.g. N meaning any of the A, T, G, C nucleotides), so the exact sequence of each variant is random, but only within the confines of the design of the experiment and oligonucleotide. This also relates to the design of the mutagenic primers, for introduction of alternate OR-type mutations in a first target region in the two or more amplification products; and in the notional division of a nucleic acid molecule of interest into different (two or more) target regions, where each target region may have a plurality of alternate mutations. This order and occurrence of these alternate OR-type mutations is random, but only within the confines of the design of the experiment and oligonucleotides. The method according to the invention differs from methods of the prior art where variant libraries may be created in a randomized manner (e.g., with the aid of E. coli XL1 red, UV irradiation, chemical mutagenesis such as deamination or alkylation, DNA shuffling, or error-prone PCR). It also differs from other site-directed mutagenesis methods that are effectively AND-type mutations, in that it encodes alternative, OR-type mutations in each variant of the library.

The mutagenic primers are preferably designed to introduce alternate mutations into one or more target regions of the two or more single stranded amplification products, which may be ordered or non-ordered. “Ordered” means that the mutations present in the variants follow a pre-defined pattern or order, for example a mutation may be introduced every 1, 2, 3, 4, 5, 10, 20, 30, 50 100 or more nucleotides; or may introduce an amino acid mutation every 1, 2, 3, 4, 5, 10, 20, 30, 50 100 or more amino acids; or may be introduced at alternate nucleotides; and/or may all be the same type of mutation (i.e. addition, substitution or deletions) or may be any pre-defined combination of addition, substitution or deletions. The ordered pattern of variants may comprise point mutations or larger mutations, and any pattern of point mutations and/or longer mutations may be introduced. Walking mutations, where each subsequent nucleotide is mutated may be preferred. In non-ordered mutations, the mutations introduced into the two or more variant amplification products may not follow any pre-defined pattern, other than the pre-requisite that the variants of said two or more amplification products do not share common mutations (i.e. the mutations in the variants are alternate, OR-type mutations).

The mutations may be designed to encode a library of protein variants having modified function. In the case of an antibody this may be improved binding properties, or for an enzyme, having a modified substrate specificity. For non-coding sequences, this may be for an optimal ribosome binding site (RBS) sequence or other regulatory element.

The nucleic acid molecule of interest may be notionally divided into target regions, wherein each target region is the site for introduction of a mutation (or mutations). A target region may be a coding or non-coding sequence, or a combination thereof. In the present invention which reduces library size by providing OR-based mutations, an amplification product of a single target region may comprise a single mutation, or multiple mutations. Two or more target sequences may be provided in a nucleic acid sequence of interest. The division is preferably carried out purely notionally, i.e., preferably no physical cleavage of the DNA starting sequence takes place (e.g., with restriction endonucleases). A target region may be any length, for example it may comprise at least 20, 30, 40, 50, 60, 70, 80, 90, 100, 120, 150, 180, 200, 500 or 1000 nucleotides, or any integer or range in-between. The limit of a target region size will typically be determined by the synthesis of the oligonucleotide primer.

A nucleic acid molecule may be divided into 2 or more, preferably 3, 4, 5, 6, 7, or 8 or more target regions. The target regions are established such that each comprises at least one mutation site. The establishment of the relative location of the target regions can be carried out in any way. Since the mutations are introduced at the mutation sites by means of mismatch positions during the amplification by PCR, the target regions are preferably established such that the mutation sites are located where the oligonucleotides (primers) bind during the PCR.

The method according to the invention pursues the goal of generating a variant library of DNA sequences with reduced complexity, such that it is feasible for all variants of the library to be screened. Preferably, the method according to the invention provides a variant library having at least 10¹, wherein each variant comprises a different mutation. The methodology is not limited by the size of the library it can create and this is therefore determined by the skilled person. Any level of similarity or difference can occur between each variant, given that they produce alternative, OR-type mutations.

In the first amplification reaction of the method of the present invention, amplification of the different variants is preferably carried out together, i.e. is not spatially separated into different amplification reactions for each mutagenic primer or subsets of mutagenic primers. However, it is envisaged that the amplification reactions using different mutagenic primers can be carried out in separate (spatially and/or temporally) reactions. The amplification products of such separate reactions could then be combined to produce an OR-type library. Therefore, in a method of the invention, a step of incubating the mutagenic primers and first primers may comprise providing each mutagenic primer and first primer in a separate incubation. The remainder of the method (steps II-IV) may then continue in the separate incubations, or the incubations may be combined for steps III and IV. Therefore, reference herein to “an incubation” includes a single vessel comprising all mutagenic primers, or multiple separate vessels each comprising a different mutagenic primer.

In step I) of a method of the present invention, a nucleic acid molecule of interest; a limited concentration of two or more mutagenic primers which each hybridise to a first strand of the nucleic acid molecule; and an excess concentration of a first primer which hybridises to a second strand of the nucleic acid molecule are brought together in an incubation in order to perform an amplification reaction. The two or more mutagenic primers are suitably provided in a limited concentration, such that they become depleted within a pre-determined number of rounds of amplification. In the incubation, reagents necessary for performance of an amplification reaction are provided, in any suitable order. Such reagents include, for example, a suitable buffer and nucleotides. Where the amplification reaction is PCR, a polymerase may be provided. A polymerase may be selected from any available polymerase, for example but not limited to, the commercially available Taq polymerase, Vent polymerase, or Pfu polymerase. In a preferred embodiment, the method of the present invention utilises a polymerase with an accurate proof reading function, and is not an error prone polymerase, such that the mutations introduced into the variants generated are substantially limited to those of the mutagenic primers used in the first PCR reaction, and not introduced through the action of the polymerase.

An incubation is maintained under suitable conditions for performing PCR. Such conditions are known in the art, and include for example denaturation at 95-98° C. for x seconds, annealing at 45-72° C. for y seconds and extension at 68-72° C. for z seconds (x, y and z being units of time, determined by the parameters of the experiment and the reagents used). It is preferable to use a high fidelity polymerase, for example Pfu, Phusion (Thermo fisher) or Q5 (New England Biolabs) polymerase, to minimise the introduction of errors from the amplification reaction. All other reagents (nucleotides, buffer, etc.) for the PCR steps are standard for PCR and known in the art.

The single stranded amplification product serves as a primer in the second round of amplification. In an embodiment, the two or more single stranded first amplification products are isolated from the first amplification reaction, for use in the second amplification reaction. Suitable methods for isolation of the two or more single stranded amplification are known and available in the art, and include for example for example gel extraction after electrophoretic separation or by gel filtration or ion-exchange chromatography or silica membrane-based purification. Alternatively, the reaction mixture of the first amplification reaction which, after the specified n umber of rounds of amplification would include the two or more first amplification products, a starting nucleic acid molecule and the excess first primer, may be used for the starting point of the second amplification without isolation of the two or more single stranded amplification products. In such an embodiment, the second primer for use in the second amplification reaction may be provided in the reaction mixture, together with a suitable polymerase and nucleotides. This incubation may serve as the basis for a second round of amplification to generate the two or more double stranded amplification products.

Suitable conditions for the second amplification reaction may be the same as or may be different to the amplification conditions used in the first amplification reaction. Where the amplification reaction is PCR, suitable conditions may be denaturation at 95-98° C. for x seconds, annealing at 45-72° C. for y seconds and extension at 68-72° C. for z seconds (x, y and z being units of time, determined by the parameters of the experiment and the reagents used). The polymerase may be the same as or different to the first PCR reaction.

The first and second amplification reactions are necessarily at least partially sequential, as the two or more single stranded amplification products are required as primers in the second amplification reaction. In a preferred embodiment, the first and second amplification steps are fully sequential, such that the desired amplification rounds of the first amplification are completed before the initiation of the second amplification reaction. However, it is envisaged that in some embodiments, there may be some overlap between the first and second amplification reactions.

In an embodiment, the method of the invention provides for a combinatorial approach, of introducing OR-type mutations into two or more target regions. In the combinatorial approach, mutations are introduced into amplification products of two or more target regions of the same nucleic acid molecule of interest such that a variant of the library will comprise a mutation in two or more target regions, neither of which mutations (i.e. the mutations in the first, second or further target region) is shared entirely by another variant. The two or more target regions of the combinatorial variant may be a single nucleic acid molecule or two or more separate nucleic acid molecules. Combinatorial variants can be created simultaneously (at the same time), sequentially (making one OR-type mutation, then another, etc) or in parallel (making all OR-type mutations separately and then combining together into one). Whichever method is used, mutations in any target region can be an OR-type mutation.

In an embodiment, amplification products of a second target region comprising an OR-type mutation are generated simultaneously, i.e. in the same first and second amplification reactions as the first target region. Therefore, in an embodiment, step I) of the method of the invention may further comprise adding a limited concentration of two or more second mutagenic primers which each hybridise to a first strand of the nucleic acid molecule to amplify a second target region. The second mutagenic primers will serve to introduce a mutation into a second target region of the nucleic acid molecule of interest, in the same way as the first mutagenic primers. Apart from the target region, the second mutagenic primers may be mutagenic primers as defined herein in relation to a first region. Step I) may also comprise adding a limited concentration of two or more third, fourth or further mutagenic primers, where it is desired to generate a mutant library of a third, fourth or further target region. In an embodiment, any second, third, fourth or further mutagenic primers are provided in the incubation prior to initiation of the first PCR reaction, such that the two or more target regions are amplified in the first PCR reaction.

Thus, the method may comprise making a variant library of a nucleic acid molecule of interest, wherein the library a population of variant nucleic acid molecules, wherein each variant comprises an alternative (Boolean OR-type) mutation in at least one target region, the method comprising:
I) incubating i) a nucleic acid molecule of interest; ii) a limited concentration of two or more first mutagenic primers which each hybridise to a first strand of the nucleic acid molecule for amplification of a first target region and a limited concentration of two or more second or further mutagenic primers which each hybridise to a first strand of the nucleic acid molecule for amplification of a second or further target region; and iii) an excess concentration of a first primer which hybridises to a second strand of the nucleic acid molecule;
II) maintaining the incubation under suitable conditions for a X number of rounds of amplification, wherein X is n+y where n is the number of amplification rounds required to deplete the mutagenic primers and y is 2 or more; to generate two or more single stranded amplification products of said first and second or further target region, each two or more single stranded amplification product comprises an alternative (OR-type) mutation in a first target region and an alternative (OR-type) mutation in a second or further target region; and
III) incubating i) the two or more said single stranded amplification products which hybridise to the second strand of the nucleic acid molecule of interest; and ii) a second primer which hybridises to the first strand of the nucleic acid molecule of interest to provide two or more double stranded amplification products comprising an alternative (OR-type) mutation in the first target region and in the second or further target region;
IV) maintaining the incubation under suitable conditions for sufficient rounds of amplification to generate the two or more double stranded amplification products wherein each double stranded amplification product comprises an alternative (OR-type) mutation in a first target region and in the second or further target regions.

Where two or more target regions are amplified in the first amplification reaction, the two or more single stranded amplification products may each contain the first, second, third or further target region. Alternatively, two or more single stranded amplification products may be generated for any single (first, second, third, fourth or further) target region, or any combination of two or more target regions. In a preferred embodiment, a two or more single stranded amplification product contains all of the target regions being amplified in the reaction.

It is appreciated that where two or more target regions are amplified in a single first PCR reaction, second primers may be provided for each target region. A second primer may be common to two or more target regions, or different second primers may be required for any two or more target regions. Any second primers used in a first amplification reaction may be as defined herein. Any further second primers are added to an incubation prior to initiation of a first amplification reaction. Two or more primers may be used for any target region.

The first amplification reaction of step II) will therefore serve to amplify said two or more target regions, preferably simultaneously. If amplified in separate first amplification reactions, the number of amplification rounds for each target region may be the same or different. These could be used separately or be pooled together to create the OR-type mutations within the library.

In step III), two or more second primers may be provided in the incubation, for each target region being amplified. If each target region is amplified separately, they may be provided to the respective separate incubations prior to initiation of the second round of amplification. Where they are being amplified in a same reaction, two or more second primers may be added to the incubation of step III) prior to the initiation of the second round of amplification of step IV).

A method of the present invention may provide one or more additional steps, for example (but not limited to) generating a double stranded nucleic acid molecule for generation of a mutant library using a method of the invention from a single stranded nucleic acid molecule; cloning the two or more double stranded amplification products into a vector, phage or other DNA; transforming a host cell with said nucleic acid to produce a library of said nucleic acid molecule of interest; selecting a desired clone from the library; isolating and purifying the variant nucleic acid molecule from the clone; cloning the nucleic acid molecule into an expression vector; and transforming a host to allow expression of the expression vector. Suitable methods are known and available to persons skilled in the art, using routine technology.

Variant nucleic acid molecules generated by a method of the present invention (a library) may be each provided within a larger, non-variant nucleic acid construct, such as a vector. Suitable vectors include an expression vector (such as chromosomal, episomal and virus-derived vectors). Examples include vectors derived from bacterial plasmids, from bacteriophage, from transposons, from yeast episomes, from insertion elements, from yeast chromosomal elements, from viruses such as baculo-viruses, papova-viruses, such as SV40, vaccinia viruses, adenoviruses, fowl pox viruses, pseudorabies viruses and retroviruses, and from combinations thereof, such as those derived from plasmid and bacteriophage genetic elements, such as cosmids and phagemids. The vector may be a virus or viral particle (such as phage or phagemid particles) displaying a polypeptide of the invention on its surface. Generally, any vector suitable to maintain, propagate or express the nucleic acid molecule in a host, may be used.

Variant nucleic acid molecules generated by a method of the present invention (a library) may be each provided as a linear nucleic acid molecule. Such a molecule may comprise the full first, second and/or further target region or a fragment thereof. Such a nucleic acid molecule may be processed further, for example operably linking to a regulatory sequence.

The variant nucleic acid molecule may be operably linked to a promoter or other regulatory sequence which controls expression of the nucleic acid. Promoters and other regulatory sequences which control expression of a nucleic acid are known in the art. The promoter may be any suitable known promoter, for example, the human cytomegalovirus (CMV) promoter, T7 bacteriophage promoter, the CMV immediate early promoter, the HSV thymidine kinase, the early and late SV40 promoters or the promoters of retroviral LTRs, such as those of the Rous Sarcoma virus (RSV) and metallothionine promoters such as the mouse metallothionine-I promoter. In an embodiment, only the minimum essential regulatory element may be required. Therefore, fragments of a promoter or regulatory sequence may be used. The promoter may comprise the minimum required for promoter activity (such as a TATA element, optionally without enhancer element) for example, the minimum sequence of the CMV promoter. Preferably, the promoter is contiguous to the nucleic acid sequence. A variant nucleic acid molecule may also be provided within a genetic construct comprising one or more of: a gene, a regulatory element and an origin of replication. A construct may be a plasmid, BAC or any other suitable construct. Indeed the two or more OR-type mutations may be provided in different places throughout a sequence. For example, the RBS sequences of multiple genes could be mutated, or the RBS sequence and coding sequence together.

A vector may include one or more expression markers which enable selection of a transfected or transformed cell. Additional control sequences such a selectable markers (e.g. antibiotic resistance, fluorescence, etc.), transcriptional control sequences and promoters, including initiation and termination sequences, may be provided.

The library may be provided contained within a population of host cells, whereby the library has first been generated and then inserted (e.g. by transformation) into the cells, suitably such that each cell contains a sequence with a different OR-type mutation. Host cells for expression of a library of the invention may include bacterial cells, such as streptococci, staphylococci, Escherichia coli, Streptomyces spp. and Bacillus subtilis; single cells, such as yeast cells, for example, Saccharomyces cerevisiae, and Aspergillus cells; insect cells such as Drosophila S2 and Spodoptera Sf9 cells, animal cells such as CHO, COS, C127, 3T3, PHK.293, and Bowes Melanoma cells and other suitable human cells; and plant cells e.g. Arabidopsis thaliana or Nicotiana spp. Suitably, the host cell is a eukaryotic cell, such as a CHO cell or a HEK293 cell. A vector may be introduced into any suitable host cell using any suitable method, for example calcium phosphate transfection, DEAE-dextran mediated transfection, microinjection, cationic-lipid-mediated transfection, electroporation, transduction, scrape loading, ballistic introduction, infection or other methods.

The method of the invention may also include preparing recombinant proteins by expression in a host cell or a cell-free system, optionally followed by isolation of the expressed protein. The method may also comprise purification of the expressed protein. Therefore, a library of the invention may provide the variants in any suitable form, for example as vectors, linear nucleic acid molecules, host cells, expression products (e.g. RNA or polypeptides). Each variant of a library may be provided in a separate spatial location, for example in a cell free system or medium. This may be in the form of an array. A library of the invention may comprise any number of distinct polypeptide variants.

A library of the invention may have reduced complexity compared to a library produced by conventional methods. For the purpose of the present invention, library complexity is the physical complexity, and is less than the theoretical complexity which is the sum of all the variants which are possible when carrying out the mutagenesis. Physical complexity corresponds to the number of variants which are actually generated when carrying out the mutagenesis. With the aid of the method according to the invention, the physical complexity of the variant library can be restricted.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

### Examples

Numerous studies have utilised asymmetric PCR for the purposes of site-directed mutagenesis (33-37). In this two-step process, the first consists of an asymmetric PCR that generates a single-stranded DNA (ssDNA) product, created by using an unequal concentration of DNA oligonucleotide primers. The lower concentration (limiting) primer encoding the mutations (termed “mutagenic primer”) becomes depleted during the early cycles of the PCR, after which the corresponding high concentration (excess) primer continues to amplify the amplicon. This generates a ssDNA product encoding all the mutations encoded by the mutagenic primers. Following purification, this product is then used as a ‘megaprimer’ in a second PCR to amplify the full-length gene encoding the library.

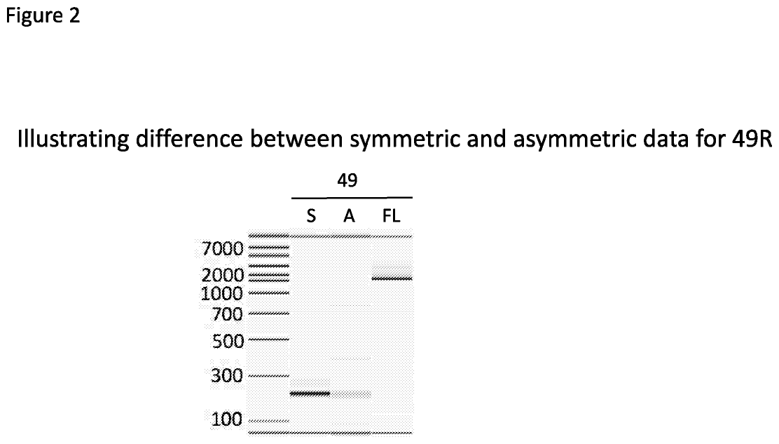

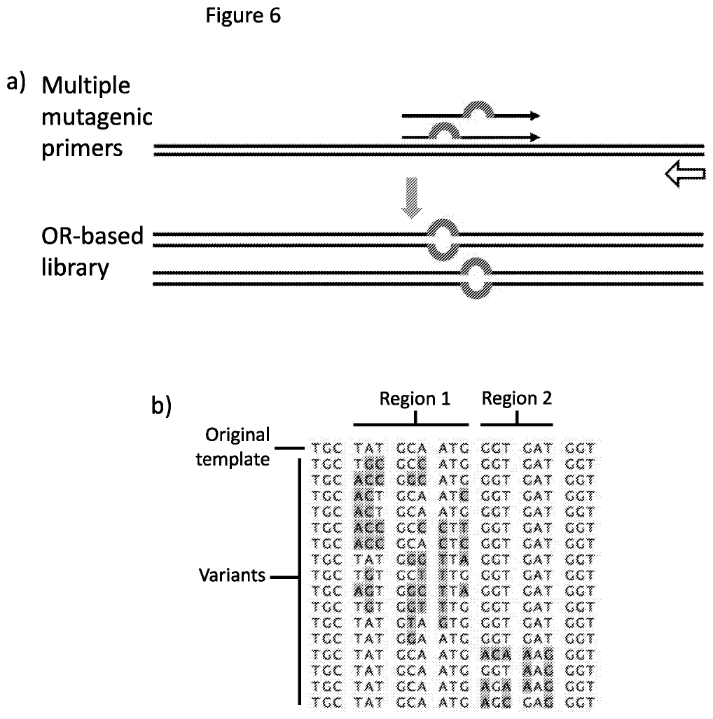

An important advantage of using asymmetric PCR is that, given that the mutagenic primers are depleted, it ensures that all mutations encoded by the primers are present in the final library (FIG. 1). This is exploited by using multiple different mutagenic primers in a single reaction, to create mutations at multiple positions in the DNA sequence. If these primers anneal to the same position in the DNA, the final library will conform to Boolean OR logic (i.e. each DNA strand encoding a mutation from {primer 1} OR {primer 2}, all such primers binding the same position are herein referred to as a “set”, FIGS. 1B and C). For example, for a simple “set” containing two mutagenic primers (FIG. 2A), the library is therefore composed of DNA strands with mutations from either primer 1 OR primer 2. It is illustrated by creating a library using 2 mutagenic primers targeting 3 and 2 amino acid codons, respectively, within the same target region (FIG. 6a). The library is therefore composed of DNA strands with mutations from either primer 1 OR primer 2. Sequencing of E. coli colonies from this library demonstrates efficient synthesis of this OR-based library (FIG. 6b) and combined with an efficient cloning method (In-Fusion, Clontech) produced 95% colonies encoding the correct full length variant sequence (not shown).

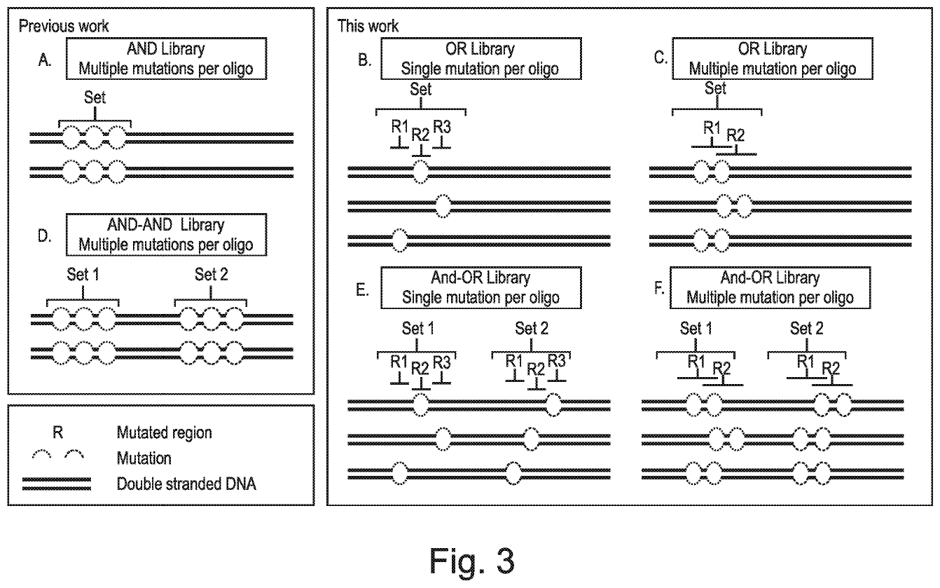

In the second scenario, combinatorial OR-type mutations (AND-OR) are created by adding an additional set of mutations (FIGS. 1E and F). This is achieved using the same method but incorporating multiple mutagenic megaprimer sets in the second PCR step. The resulting library encodes OR-type mutations for each set added, i.e. each member of the library encodes an OR-type mutation from set 1 and an OR-type mutation from set 2 (i.e. AND-OR mutations, FIG. 3B).

Here, an experimental implementation of the Boolean OR function for library generation for directed evolution (e.g. mutate {codon/region 1} OR {codon/region 2} OR {codon/region 3}, etc.) is provided. The effect of mutating multiple regions (codon or set of codons) on library size in this way is therefore additive and not multiplicative/exponential. The data demonstrates that this strategy can be employed to reduce the library size significantly (often by many orders of magnitude), as well as decreasing its complexity, enabling the mutation of a larger number of regions in the same library. This has the highly desirable effect of significantly reducing the overall size of the library, while still testing all the desired codons and mutations.

### Creation of OR-Based Libraries for Directed Evolution

The benefit of OR-type libraries is demonstrated through two approaches using monoamine oxidase-N (MAO-N) as the exemplar enzyme target.

Firstly, a large-scale mutagenesis approach was adopted, mutating 276 amino acids of MAO-N (of a total 495 amino acids); these account for every residue known to exhibit secondary structure. Our approach permitted several (typically up to 12) amino acids to be mutated in a single library without the combinatorial explosion that would occur when using AND-type libraries. Multiple variants were identified with increased k_{cat} towards both native and non-native amine substrates, including novel activity for new substrates.

The methodology was to create combinatorial OR-based libraries for 4 residues surrounding the substrate binding pocket of MAO-N, a library which still encodes mutations for all 4 residues but reduces the size of the library (and hence screening effort) 256-fold. In both instances, the OR-type mutations greatly reduce the overall size of the library and consequently the amount of screening that needs to be performed.

Second, combinatorial OR-type libraries were created (effectively encoding AND-OR mutations) for a Combinatorial Active-Site Saturation Test (CASTing (39, 40)). Using this approach, 10 active site residues were mutated simultaneously, such that many different combinations of 2 residue mutations were tested in one library. These AND-OR mutations reduced the library size 4.4×10¹⁰-fold, compared to simultaneous randomisation of all residues (AND mutations). This enabled the screening of a library with more diverse mutations compared to conventional methods, and the rapid discovery of a new variant exhibiting activity towards 2 novel substrates.

The method was utilised for a large mutagenesis study for the directed evolution of MAO-N. MAO-N is an important industrial biocatalyst that oxidises a variety of primary, secondary and tertiary amines (33-35, 56, 30, 55, 57, 58). Wild type MAO-N exhibits very low activity (k_{cat}=0.17 min⁻¹) towards the primary amine □-MBA, however previous directed evolution studies have generated a variant (Ile246Met/Asn336Ser/Met348Lys/Thr384Asn/Asp385Ser, termed D5 (37, 38, 30) with a k_{cat} of 154 min⁻¹. This is still significantly lower than that of the wild type MAO-N for several primary amine substrates (262-394 min⁻¹) that are believed to be similar to the native substrates (rates referred to as ‘wild type speed’). This k_{cat} is roughly half that of the wild type for its native (or most active) primary amine substrates; hence we devised a strategy to seek variants with a “wild type speed” k_{cat} towards the non-native substrate □-MBA.

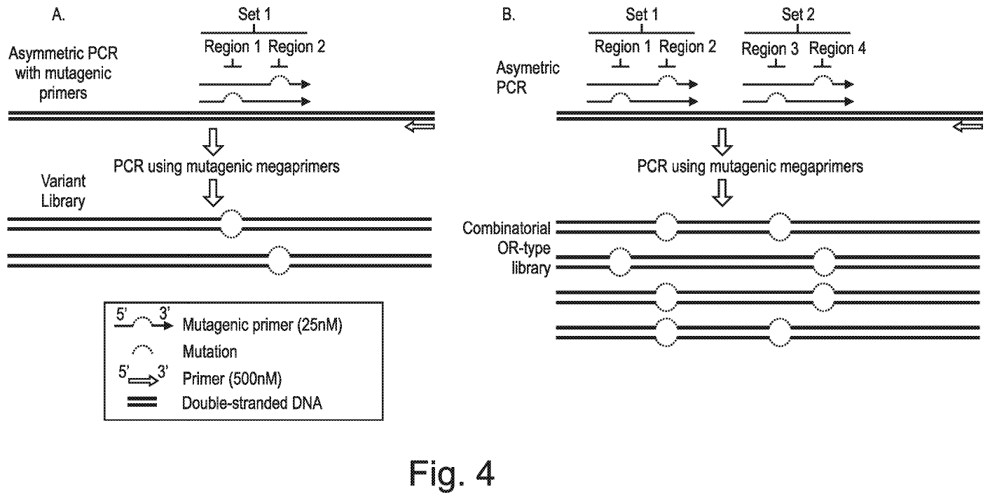

In this work no more than 3 amino acids were encoded by a single mutagenic primer, hence keeping <2000 genetic combinations for each variant (<150 amino acid combinations). However, following the present method, multiple mutagenic primers were utilised for each library, permitting up to 12 amino acids to be mutated in each sample (mutating 3 OR 3 OR 3 OR 3 amino acids). Consequently, this produced total libraries of <8000 genetic combinations (2000+2000+2000+2000). Mutation of the same residues simultaneously (AND rule, 2000×2000×2000×2000) produces a library of 1.6×10¹⁴. Hence, the method has reduced the library size 2.0×10¹⁰-fold. In short, the invention provides an effective means to screen large numbers of mutations in a controlled manner, whilst simultaneously reducing the library size to one that is possible to screen fully. For MAO-N the screening was a colony-based colorimetric screen, measuring amine oxidation through production of H₂O₂ (FIG. 4).

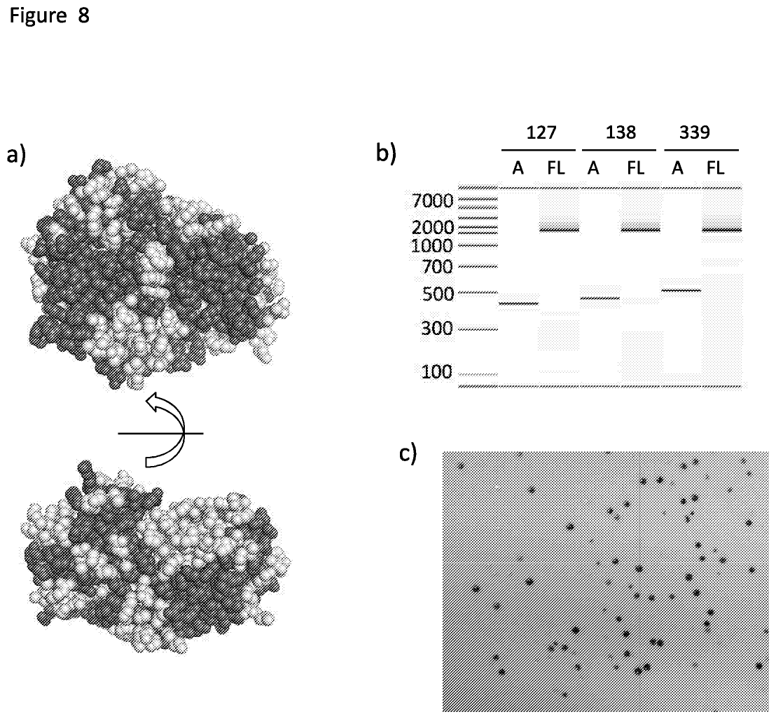

Given the reduction in library size that the present invention enables, it is possible to screen a large number of mutations of MAO-N and screen them for changes in catalytic activity. In total, 276 amino acids (from a total of 495 amino acids) were mutated using specified mixed base codons (FIG. 8). These were targeted throughout the protein sequence.

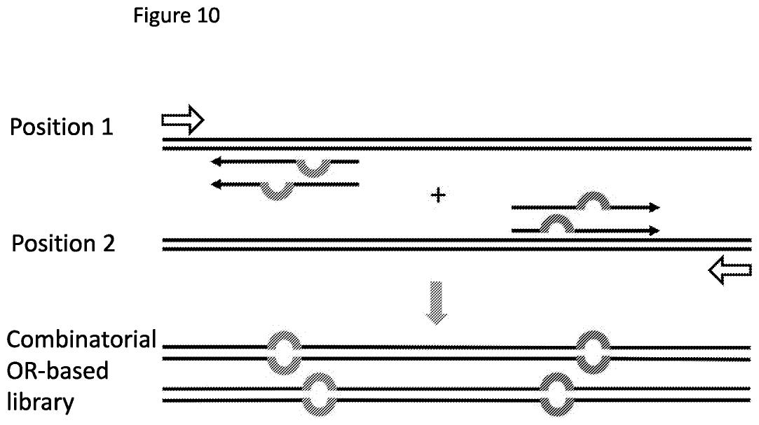

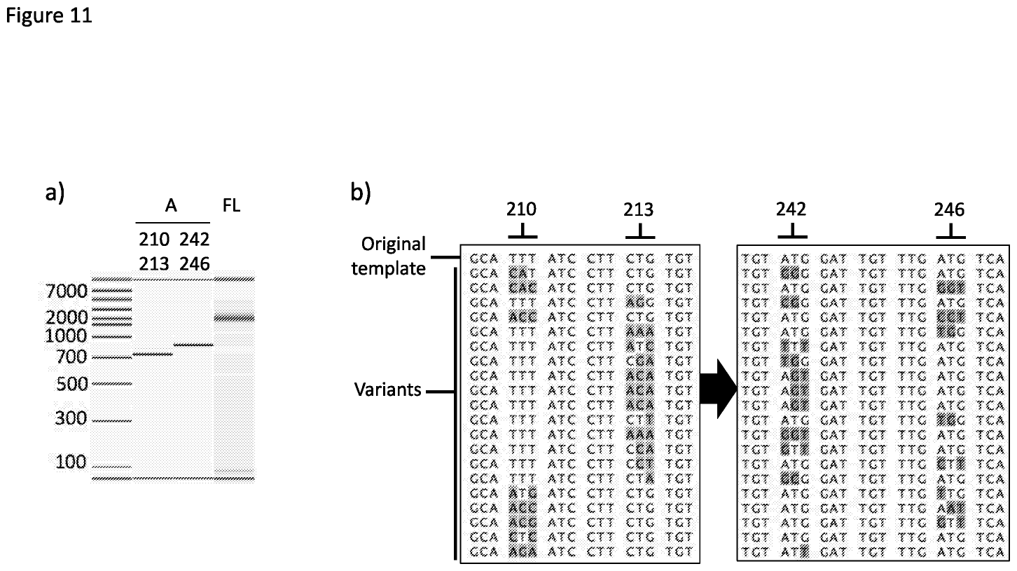

### Combinatorial OR-Based Libraries

Whereas the original method (above) uses mutagenic primers targeted to a single contiguous target region, the method can be extended to create OR-based libraries at multiple target regions simultaneously (FIG. 10). When multiple mutagenic primers are used at each target region, the library produced encodes OR-based mutations at both regions. Although not testing all possible combinations, this method enables the testing of different combinations of mutations. This will be particularly useful when wishing to search for an optimal combination of mutations from a pool of many possible candidates. As an example, we selected 4 amino acids (F210, L213, M242 and M246) that are positioned at the entrance to the active site of MAO-N and have previously been mutated together, but not in such a way to test different combinations between the 4 residues (30). The NNK codon was utilised (32 genetic combinations encoding all 20 amino acids) for full randomisation of each residue. In this example, F210 and L213 comprised target region 1 and M242 and M246 comprised target region 2 (with each codon mutated using a separate oligo). Consequently, two megaprimers of different length were produced in the asymmetric PCR step. These were then used to create a single full-length MAO-N library (FIG. 11). Sequencing of selected E. coli colonies from this library showed that OR-based mutations had been efficiently created at created at both target regions. We demonstrate that this approach reduces to total size of the library 256-fold from 1.05×10⁶ to 4096 genetic combinations, suitable for the colony-based MAO-N screening (Table 1).

TABLE 1
An illustration of how OR-based libraries can greatly reduce the size
of variant libraries. If four amino acids are to be mutated using the
NNK codon (32 possible genetic combinations, encoding all 20 amino acids), mutation
of each residue simultaneously (1 AND 2 AND 3 AND 4) produces >10⁶ possible
combinations. However, mutating the same residues in different combinations
of two ((1 OR 2) AND (3 OR 4)) produces 4096 combinations, a 256-fold
reduction in library size. Note, each of these libraries is prepared
in the same PCR procedure using asymmetric PCR.

| | Variant | Number of mutations | Number of genetic |
|---|---|---|---|
| Residue(s) mutated | codon(s) | per sequence | combinations |
| 1 | NNK | 1 | 32 |
| 2 | NNK | 1 | 32 |
| 3 | NNK | 1 | 32 |
| 4 | NNK | 1 | 32 |
| 1 AND 2 AND 3 AND 4 | NNK | 4 | 1048576 |
| (1 OR 2) AND (3 OR 4) | NNK | 2 | 4096 |
| (1 AND 2) OR (3 AND 4) | NNK | 2 | 2048 |
| 1 OR 2 OR 3 OR 4 | NNK | 1 | 128 |

In this study, every amino acid in MAO-N D5 possessing secondary structure was mutated according to our mutagenesis design (see below), totalling 276 amino acids. Each mutagenesis region (encoded by a single mutagenic primer) was limited to <3 amino acids each using our design of ambiguous codons (scenario C in FIG. 1, Table 1). Following the GeneORator approach, multiple mutagenic primers for consecutive regions were incorporated in each library. In one example (FIG. 3), 4 regions were created to mutate 11 consecutive codons. Simultaneous mutation of all 11 codons together (AND-type mutations) would create over 10¹¹ genetic combinations, whereas a corresponding OR-type library encodes 5136 combinations, a 1.1×10⁸-fold reduction.

Given that protein secondary structure often follows a regular binary pattern of polar (P) and non-polar (NP) residues (e.g. amphiphilic helices can follow a P-NP-P-P-NP-NP-P pattern) one strategy to ensure that the majority of the searched sequence space encodes proteins with similar secondary structure is to follow this semi-conservative binary pattern (40), such that tertiary structure is more-or-less conserved. Hence, a codon mutagenesis approach was devised to increase the proportion of functional protein variants by binary patterning (Table 2). The strategy for mutating polar to alternative polar residues, and non-polar to other non-polar residues is intended to limit our sequences to that of more ‘functional sequence space’. For example, when Leu is the starting amino acid, we mutated it using the NTN codon (N=A, T, G or C) to encode Phe, Leu, Ile, Met or Val.

The large-scale mutagenesis strategy is guided by the understanding that amino acids throughout the protein structure, often distal to the active site, have a significant effect on the efficiency of catalysis (k_{cat}/Kₘ or k_{cat})⁽⁴¹⁾. Hence creating mutations throughout the protein structure will enable us to detect those variants with significantly increased k_{cat} for a panel of native and non-native amine substrates.

### MAO-N Improved Variants to Non-Native Amine Substrates

The tertiary structure of MAO-N D5 is known⁽³⁶⁾, which facilitated the identification of all residues displaying secondary structure. In total 276 amino acids were selected and mutated using OR-type libraries. Using the previously described colony-based screening method to analyse oxidase activity by detection of hydrogen peroxide (35, 42), every OR-type library was screened using α-MBA, attempting to improve the k_{cat} towards this non-native substrate. For each library, the top (fastest) colonies were selected and the DNA sequenced. Sequences that showed a clear selection for a new variant (e.g. a mutation selected multiple times) were characterised.

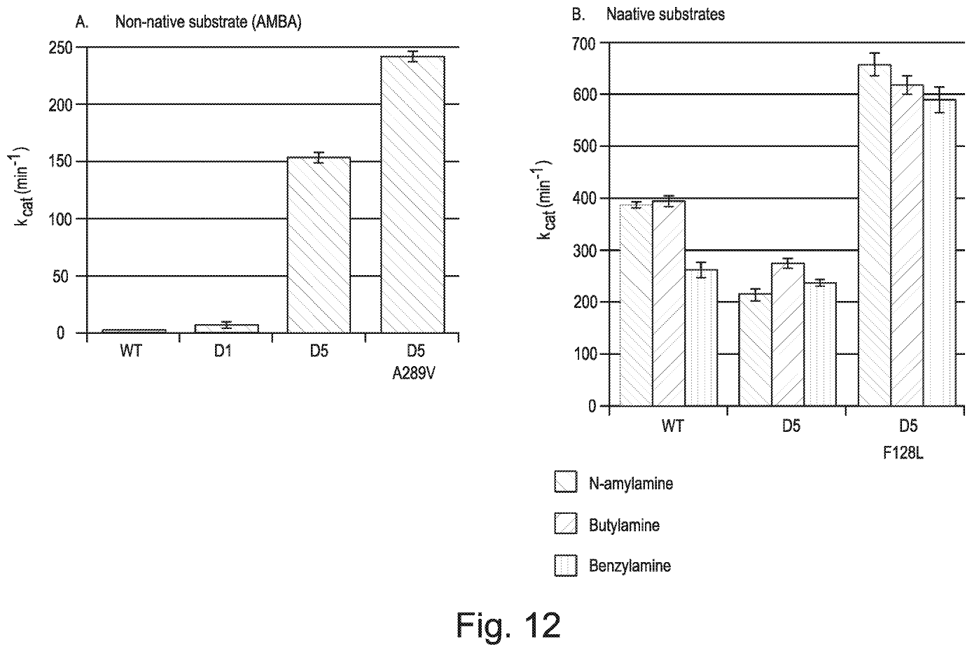

Four variants were identified with an elevated k_{cat} compared to the D5 variant (FIG. 12). One variant, A289V (k_{cat}=242 min⁻¹), exhibited a 1.6-fold increase to that of its parent D5 and a 1210-fold increase to that of the wild type.

### MAO-N Activity to Native Primary Amine Substrates

In addition to characterising MAO-N variants towards α-MBA, we also tested the OR library against the native WT substrates, where several variants also exhibited increased activity (Table 4 and 5). Interestingly, the best α-MBA variant (A289V) was not the fastest towards these substrates, but F128L was faster for all three native substrates. F128L activity to N-amylamine (AA, 655 min⁻¹) is the highest k_{cat} published for MAO-N for any substrate to date, 1.7-fold higher than the WT and 3-fold faster than its parent D5 variant.

### MAO-N Activity to Novel Substrates

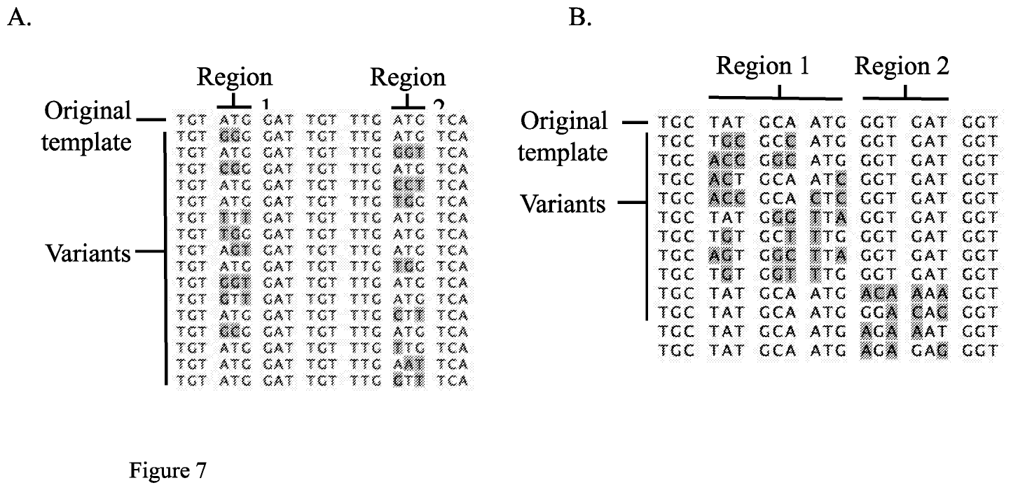

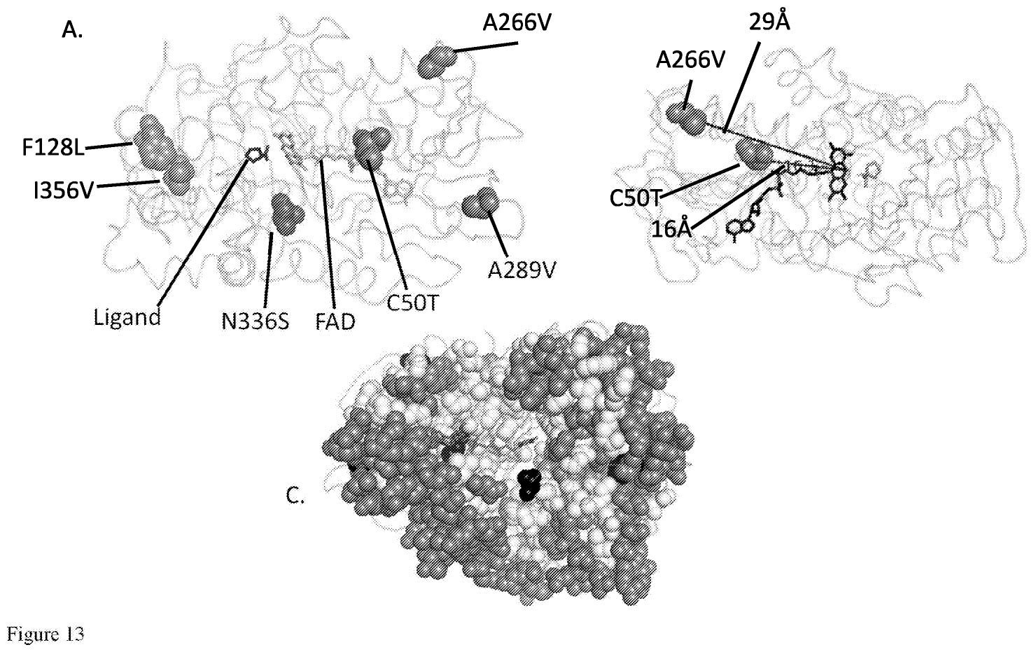

No published MAO-N variants to date (including WT and D5) exhibit detectable activity towards the primary amine cyclohexylamine (CHA). However, activity was detected (k_{cat}=17 min⁻¹) for one of the variants (A266V). To improve this activity, the strategy was to create combinatorial mutations at multiple positions (as described in section 2.1). Combinatorial OR-type mutations were tested for mutations selected from the first round of screening, which identified a double mutant C50T A266V with improved activity for CHA (k_{cat}=38 min⁻¹). Given that neither C50 nor A266 are positioned in the active site (29 Å and 16 Å from the FAD catalytic amine, respectively, see FIGS. 7 and 13, and Tables 4 and 5), such data show that residues distal to the active site also contribute specificity for substrates and mutagenesis of these residues can yield variants with activity towards novel substrates (43).

### Active Site Mutagenesis Using Combinatorial OR-Type Mutations

The benefit of OR-type mutations becomes more significant when applied to screening multiple mutations combinatorially, given its additive nature prevents the combinatorial explosion of mutation combinations associated with conventional AND-type mutations. To demonstrate this, OR-type combinatorial mutations were created for 10 amino acids in and around the active site of MAO-N for CASTing (scenario E in FIG. 1). The residues were divided into two sets (each containing 5 amino acids) and the megaprimers for each set were pooled together in the second PCR step to create combinatorial OR-type mutations (effectively AND-OR mutations, FIG. 3B). Each amino acid was mutated using the NNK codon (32 possible combinations encoding all 20 amino acids). Consequently, in this CASTing library every amino acid substitution for all 5 amino acids in set 1 was mutated with every amino acid substitution in set 2. Mutation of all 10 amino acids together (AND-type library) would create 1.1×10¹⁵ combinations (=32¹⁰), whereas our AND-OR library encodes 25600 combinations, a 4.4×10¹⁰-fold reduction in DNA library size. Alternatively, to recreate each of these mutation combinations without AND-OR libraries would require the synthesis of 25 separate libraries.

Screening of the AND-OR CASTing library identified a new variant (A209S/L245C) with novel activity to two non-native substrates (1-(3-bromophenyl)ethan-1-amine and 1-(3-methoxyphenyl)ethan-1-amine, Supplementary material). These mutations were encoded in position [1] and [5] in sets 1 and 2 (respectively), a combination that could not have been predicted by structural or sequence analysis. This therefore demonstrates the benefit of combinatorial OR-type mutations (AND-OR) for the screening of many mutation combinations, significantly reducing the experimental effort of creating all the different mutations separately. Effectively this strategy permits the screening of a more diverse number of mutation combinations quickly in the search for improved and novel enzyme function.

### Discussion

In the majority of directed evolution studies, the size of the library is a significant limiting factor in the search for improved variants. Often there are a number of candidate amino acids that the experimenter would like to test, however mutating even a small number of these simultaneously rapidly creates libraries too large to screen by even high-throughput methods. Here we introduce a method devised to create OR-type libraries. This enables the mutation of multiple amino acids in a single sample without testing all residues simultaneously with each other. The effect of these mutations is therefore additive (the OR rule in Boolean logic), a feature that reduces the overall library size dramatically, often by several orders of magnitude. Consequently, this provides the opportunity to mutate a greater number of positions in a target sequence, greatly facilitating the search for “hits” from an otherwise vast and untestable sequence space.

In two examples, the technology was utilised to create two different types of OR-based libraries. Initially, the method was used to mutate a total of 276 amino acids in a single gene sequence (MAO-N). By mutating no more than 3 amino acids per mutagenic primer, and through use of multiple primers per sample, we reduced the size of each of the libraries by up to 2×10¹⁰. This methodology was fundamental in the strategy of mutating large numbers of residues, while also making it possible to screen these libraries using a colony-based activity assay.

In the second example, combinatorial OR-based libraries were created to randomise mutations at 4 amino acids positions. This enables us to test the effect of different combinations of these mutations in the search for an optimal combination, which would not be possible to do using other mutagenesis methods without creating very large libraries (for example >1×10⁶ in size) or using significantly more resources.

In this study a methodology to create a novel type of variant library has been demonstrated, whereby multiple discrete DNA regions can be mutated in an OR-type fashion. The result is that each region contributes an additive effect to the total library size (Boolean OR logic), in contrast to conventional site-directed mutagenesis methods (utilising AND logic) where multiple mutations create a combinatorial explosion (for example, 20n protein variants when fully randomising amino acids).

Here, OR-type libraries have been exploited to implement a novel mutagenesis scheme based on the binary patterning feature of protein secondary structure. An ambiguous codon design strategy has been devised and used to mutate every amino acid in the secondary structure-containing regions of MAO-N. This design sought to conserve the pattern of polar and non-polar residues present in the MAO-N sequence, an approach predicted to improve the proportion of variants with the secondary structure required to create the tertiary fold required for catalysis. Taken together, the mutagenesis methodology and library design enabled large-scale mutagenesis studies to improve the search of ‘functional sequence space’, in a way that is not economic (nor feasible) using existing approaches. Regardless of the codon mutagenesis strategy, the data demonstrated that the experimental approach was efficient at generating the designed OR-type mutations for screening. The approach is therefore broadly applicable to any mutagenesis study where multiple mutations are to be created and screened.

In this study several residues were discovered distal to the active site that conferred an increase in k_{cat} in a manner that was not predictable from knowledge of amino acid sequence, tertiary structure or catalytic mechanism. Given that these mutations are not predicted to alter the protein's basic secondary structure, it is expected that these mutations improve activity through alteration in protein dynamics during catalysis, rather than via major ground-state structural changes (see also (44, 45)).

Given the knowledge of which variants had been screened in each library we obtained sequence-activity data for every library that was screened. This provides an insight into the in vitro selection of every amino acid mutated in the study (FIGS. 7, and 13; Tables 4 and 5). We discovered strong selection at 120 residues, where the amino acid encoded in the WT was invariant. Conversely, many amino acids were tolerant of several different mutations whilst still maintaining good catalytic activity. In total, of those assessed, 53 residues could encode one other residue, 44 could accommodate two mutations and 50 could accommodate three or more mutations. High-frequency selection for a new mutation was discovered for 9 amino acids and each of these mutations were characterised (above). We also found that strong selection for WT residues was more frequently observed for amino acids closer to the protein core and to the FAD cofactor. Combining these data with that of the mutations that increase k_{cat} provides important information on the selection pressure exerted on every residue in the secondary structure during our screening. Interestingly, combining the four different mutations together did not yield an additive effect; no double mutants exhibited an increased k_{cat} for α-MBA above the single mutants, thus illustrating the highly epistatic nature of this protein's landscape.

There is widespread interest in exploiting in silico learning algorithms for biological applications. Machine learning provides the opportunity to learn complex sequence-activity relationships and to predict variants with improved fitness (46). Principled search algorithms like “Protein sequence activity relationships” (ProSAR) have been used to help engineer enzymes by creating partial least square (PLS) regression models, and recent updates may accommodate epistatic interactions between two residues (47, 48, 49). We envisage that improved technology in DNA library synthesis and ‘deep mutational scanning’ (50, 51) will empower learning algorithms to predict proteins with improved fitness for a variety of directed evolution applications. Given the complexity of protein sequence-activity relationships, especially the importance of epistasis, learning algorithms require the ability to design specific yet complex DNA libraries for screening. GeneORator is capable of creating these libraries in a way that does not create the combinatorial explosion associated with conventional libraries and is a powerful tool in the rapid discovery of new biocatalysts with improved and novel activity.

Given the uses of OR-based libraries stated above, there are also many different permutations and elaborations to the method that deems it useful for a variety of applications. These could include alanine scanning (52, 21), whereby many candidate residues could be mutated to alanine in isolation but within the same sample (OR-type alanine mutations). In this way only one library need be created to target many amino acids (if using suitably long mutagenic primers), significantly reducing the preparation time and costs of creating each mutation in parallel. The methodology is also envisaged to be a valuable tool to supplement existing methods for libraries and screening, it could be used alongside fully exhaustive mutation libraries (for example in antibody discovery) to improve a library further (for example in antibody maturation or optimisation of any other desirable function or characteristic). These methods can therefore be wholly complementary.

### Materials and Methods

### Design of Oligonucleotide Primers for OR-Type Libraries

The MAO-N D5 gene (Uniprot ID: P46882) was designed using GeneGenie (61) and synthesised using the SpeedyGenes gene synthesis method, as previously described (62, 63). In the design of OR-type libraries, first the number of target regions and the number of codons to be mutated was identified (typically up to four target regions, each containing up to three codon mutations). Flanking sequences to these target regions were selected, such that the annealing temperature (Tₘ) was predicted to be 60° C. at both the 5′ and 3′ termini. The relevant ambiguous codons were then inserted into the oligonucleotide sequence, depending on the amino acids present in the parent D5 sequence (see Table 1). One mutagenic primer was designed for each target region, such that a set of primers encoded the same 5′ and 3′ flanking sequences but each different target region mutations. Corresponding end PCR (non-mutagenic) primers were also designed with a predicted annealing temperature (Tₘ) of 60° C. for the 5′ and 3′ termini of the gene.

### Synthesis of OR-Type Libraries

DNA oligonucleotides were synthesised by Integrated DNA Technologies. For asymmetric PCR, the reaction contained 25 nM mutagenic (limiting, forward read) primer and 500 nM end (excess, reverse read) primer, with 0.5 ng μL⁻¹ template (MAO-N D5), 0.2 mM dNTP mix, Q5 reaction buffer and 0.02 U μL⁻¹ Q5 hot-start high-fidelity polymerase (New England Biolabs) in 50 μL total volume. The PCR consisted of denaturation at 98° C. for 30 s, then 25 cycles of 98° C. for 20 s, 60° C. for 20 s and 72° C. for 40 s. PCR products containing ssDNA were purified using a PCR purification kit (Qiagen).

For symmetric PCR to assemble the full gene, the ssDNA PCR product from asymmetric PCR was used as the megaprimer (reverse read) together with the relevant end primer (forward read). The reaction contained 16.5 μL megaprimer, 500 nM end primer and other reagents as above. The PCR consisted of denaturation at 98° C. for 30 s, then 25 cycles of 98° C. for 30 s, 60° C. for 20 s and 72° C. for 40 s. For combinatorial OR-type libraries, megaprimers were created for each set of mutations and pooled together in the in the PCR above [see MIE CHAPTER]. PCR products were visualised and purified by gel electrophoresis and gel extraction (Qiagen kit).

### Screening for MAO-N Activity

Purified libraries were ligated into a linearised expression vector (pET16b, Novagen) using the In-Fusion cloning kit (Clontech), following the manufacturers' protocol. Ligation reactions were transformed into E. coli competent cells (T7 express, New England Biolabs) and spread onto an LB agar with 100 μg mL⁻¹ ampicillin covered with a Hybond-N membrane (Amersham biosciences). Following incubation overnight at 30° C., the membrane containing single colonies was transferred to an LB agar plate (100 μg mL⁻¹ ampicillin and 1 mM IPTG) and incubated for two hours at 30° C. Oxidase activity was then assayed following the protocol outlined previously (33, 54). Briefly, the membrane containing colonies was transferred to a membrane soaked in 0.1 mg mL⁻¹ HRP (Sigma) and 100 mM potassium phosphate pH 7.7 for 30 min (the prescreen). Colonies were then transferred to membrane soaked in 0.1 mg ml⁻¹ HRP, DAB (Sigma), 2.5 mM α-methylbenzylamine (Sigma) and 100 mM potassium phosphate pH 7.7. Oxidase activity was observed by the formation of a brown DAB precipitate.

Colonies that exhibited the fastest colour change were picked and inoculated into LB (100 μg mL⁻¹ ampicillin), grown overnight (37° C., 180 rpm) and the plasmids were extracted using a plasmid miniprep kit (Qiagen). Sequencing of variants was performed using Sanger sequencing (Eurofins).

### Expression and Purification of MAO-N

Selected variants were overexpressed by BL21 (DE3) E. coli strain in 700 mL of LB medium with 100 μg mL⁻¹ ampicillin. 0.5 mM of IPTG was introduced to the culture when OD₆₀₀ reached 0.6 and the culture was incubated at 25° C., 180 rpm. Cells were harvested after 16-20 hours and purified using 5 mL Histrap FF crude column (GE Healthcare) with an AKTA Explorer 100 protein purification system as described (55).

### Liquid Phase Kinetic Assay

A range of amine stock solutions including α-methylbenzylamine, N-amylamine, butylamine, benzylamine or cyclohexylamine (all from Sigma) were prepared in 0.1 M potassium phosphate pH 7.7. The final concentration range of the substrate was between 0.5 mM to 100 mM. A colourimetric assay solution was made up by dissolving Pyrogallol red (0.3 mM final concentration) in 0.1 M potassium phosphate, pH 7.7. The assay was conducted by combining 35 μL of substrate solution, 50 μL of Pyrogallol red solution and 5 μL of horseradish peroxidase (1 mg mL⁻¹) in a flat bottom 96-well plate and started by adding 110 μL of purified MAO-N. Assay progress was monitored at 550 nm at 25° C. in a Molecular Devices Spectramex M2 plate reader. The data were analysed using Prism7 (GraphPad) which was also used to calculate kinetic parameters such as k_{cat}, kₘ and Vₘₐₓ.

TABLE 2
The mixed-base codon design used in this study. Each degenerate codon
was designed to encode amino acids that maintained the major physical
properties of the original amino acid in MAO-N. For example, Leu was
mutated to other non-polar amino acids using the NTN codon and Ser was
mutated to four other polar residues (plus itself) using the WVY codon.

| | | | | | Amino |
|---|---|---|---|---|---|
| | Physicochemical | Mixed base | Degenerate | Genetic | acids |
| Amino acid | property | sequence | codon | combinations | encoded |
| Phe, Leu, | Non-polar | (ATGC) T | NTN | 16 | Phe, Leu, |
| Ile, Met, Val | | (ATGC) | | | Ile, Met, Val |
| Asn, Gln, | Polar, Acidic | (ACG) A | VAN | 12 | Asn, Gln, |
| Asp, Glu, | and Basic | (ATGC) | | | Asp, Glu, |
| His, Lys | | | | | His, Lys |
| Ala | Non-polar | G (CGT) | GBN | 12 | Ala, Gly, Val |
| | | (ATGC) | | | |
| Arg | Basic and Polar | (AC)(AG)(AT) | MRW | 8 | Arg, His, |
| | | | | | Lys, Gln, |
| | | | | | Asn, Ser |
| Gly | Non-polar, | (AG) (CG) A | RSA | 4 | Gly, Ala, |
| | Basic and Polar | | | | Arg, Thr |
| Pro | Non-polar | CCT | CCT | 1 | Pro |
| Ser, Tyr, | Polar | (AT) (ACG) | WVY | 12 | Ser, Tyr, |
| Cys, Thr, | | (CT) | | | Cys, Thr, |
| Asn | | | | | Asn |
| Trp | Non-polar and | T (GT) (GT) | TKK | 4 | Trp, Phe, |
| | Polar | | | | Leu, Cys |

TABLE 3
Measured k_{cat} (min⁻¹) values of selected MAO-N variants (those
of importance are emphasised in bold). Full kinetics data is shown
in Supplementary material. Abbreviation: N.D.—none detected.

| | | Substrate (k_{cat} min⁻¹) | | | | | | |
|---|---|---|---|---|---|---|---|---|
| MAO-N variant | Reference | AMBA | AA | BTA | BZA | CHA | BPEA | MPEA |
| WT | {Alexeeva 2002} | 0.17 | 387 | 394 | 262 | N.D. | | |
| D1 N336S | {Alexeeva 2002} | 8.0 | | | | N.D. | | |
| D5 (parent | {Dunsmore 2005} | 154 | 218 | 275 | 237 | N.D. | N.D. | N.D. |
| sequence) | | | | | | | | |
| D5 A289V | This study | 242 | | | | N.D. | | |
| D5 F128L | This study | 217 | 655 | 616 | 589 | N.D. | | |
| D5 C50T | This study | | | | | 38 | | |
| A266V | | | | | | | | |
| D5 A209S | This study | | | | | | 100-200 | 100-200 |
| L245C | | | | | | | | |

TABLE 4
Kinetic parameters of MAO-N wild type, D5 and selected variants for the non-native substrate α-methylbenzylamine.
A. The variant A289V (highlighted bold) with highest k_{cat} exhibits a 1210-fold increase in activity to the wild type
and 1.6-fold increase to the parent D5. B. Improvement in k_{cat} of MAO-N variants, where the A289V variant has roughly
equal k_{cat} for α-methylbenzylamine (α-MBA) to that of the wild type to benzylamine (BZA).

| | | | | | | | | Improvement |
|---|---|---|---|---|---|---|---|---|
| | | | Kₘ | Vₘₐₓ | k_{cat} | K_{cat} | k_{cat}/Kₘ | over wild |
| Variant | Reference | Kₘ (mM) | Std Error | (U/mg) | (min⁻¹) | Std Error | (mM min⁻¹) | type |
| Wild type | {Alexeeva 2002} | ND | | | 0.17 | | | — |
| D1 N336S | {Alexeeva 2002} | 0.4 | | | 8.0 | | | 40-fold |
| D5 (parent | {Dunsmore 2005} | 4 | 0.5 | 2.8 | 154 | 7.7 | 38 | 771-fold |
| sequence) | | | | | | | | |
| D5 A289V | This study | 3.8 | 0.3 | 4.4 | 242 | 7.1 | 63 | 1210-fold |
| D5 I356V | This study | 5.6 | 1.4 | 4 | 220 | 24 | 40 | 1099-fold |
| D5 F128L | This study | 4.7 | 0.5 | 3.9 | 217 | 9.7 | 46 | 1086-fold |
| D5 A266V | This study | 6.7 | 0.9 | 3.7 | 203 | 14 | 30 | 1017-fold |

TABLE 5
Comparing the activity of MAO-N variants to primary amine substrates. A. Table comparing
the k_{cat} (min⁻¹) of selected MAO-N variants (ND = none detected), increased
activity compared to the wild type are highlighted (bold). B. Increase in activity
to these substrates, from the wild type to D5 variant and our best variant F128L.

| | N-amylamine (AA) | | Butylamine (BTA) | | Benzylamine (BZA) | | Cyclohexylamine (CHA) | |
|---|---|---|---|---|---|---|---|---|
| | k_{cat} | Std | k_{cat} | Std | k_{cat} | Std | k_{cat} | Std |
| | (min⁻¹) | Error | (min⁻¹) | Error | (min⁻¹) | Error | (min⁻¹) | Error |
| WT | 387 | 5.3 | 394 | 5.7 | 262 | 13 | ND | |
| D5 | 218 | 5.9 | 275 | 8.3 | 237 | 5.6 | ND | |
| D5 C50T | 292 | 7.5 | 367 | 16 | 323 | 7.4 | ND | |
| D5 F128L | 655 | 21 | 616 | 18 | 589 | 21 | ND | |
| D5 A266V | 355 | 6.6 | 429 | 9.2 | 424 | 5.1 | 17 | 2.1 |
| D5 I356V | 262 | 26 | 366 | 5.7 | 410 | 9.1 | ND | |
| D5 F128L | 528 | 14 | 377 | 10 | 499 | 8.1 | 14.8 | 1 |
| A266V | | | | | | | | |
| D5 C50T | 203 | 10 | 262 | 14 | 247 | 5 | 28 | 5.4 |
| A266V | | | | | | | | |

### REFERENCES

1. Arnold F H, Georgiou G. Directed Enzyme Evolution [Internet]. Vol. 230. New Jersey: Humana Press; 2003 [cited 2016 Jan. 26]. Available from: http://link.springer.com/10.1385/1592593968
2. Cheng F, Zhu L, Schwaneberg U. Directed evolution 2.0: improving and deciphering enzyme properties. Chem Commun. 2015 Jun. 2; 51(48):9760-72.
3. Denard C A, Ren H, Zhao H. Improving and repurposing biocatalysts via directed evolution. Curr Opin Chem Biol. 2015 April; 25:55-64.
4. Dougherty M J, Arnold F H. Directed evolution: new parts and optimized function. Curr Opin Biotechnol. 2009 August; 20(4):486-91.
5. Jäckel C, Kast P, Hilvert D. Protein Design by Directed Evolution. Annu Rev Biophys. 2008; 37(1):153-73.
6. Lutz S. Beyond directed evolution—semi-rational protein engineering and design. Curr Opin Biotechnol. 2010 December; 21(6):734-43.
7. Cobb R E, Si T, Zhao H. Directed Evolution: An Evolving and Enabling Synthetic Biology Tool. Curr Opin Chem Biol. 2012 August; 16(3-4):285-91.
8. Cobb R E, Sun N, Zhao H. Directed evolution as a powerful synthetic biology tool. Methods. 2013 Mar. 15; 60(1):81-90.
9. Dalby P A. Strategy and success for the directed evolution of enzymes. Curr Opin Struct Biol. 2011 August; 21(4):473-80.
10. Copp J, Hanson-Manful P, Ackerley D, Patrick W. Error-Prone PCR and Effective Generation of Gene Variant Libraries for Directed Evolution. In: Gillam E M J, Copp J N, Ackerley D, editors. Directed Evolution Library Creation [Internet]. Springer New York; 2014 [cited 2016 May 19]. p. 3-22. (Methods in Molecular Biology). Available from: http://dx.doi.org/10.1007/978-1-4939-1053-3_1
11. Cadwell R C, Joyce G F. Randomization of genes by PCR mutagenesis. Genome Res. 1992 Aug. 1; 2(1):28-33.
12. McCullum E O, Williams B A R, Zhang J, Chaput J C. Random Mutagenesis by Error-Prone PCR. In: Braman J, editor. In Vitro Mutagenesis Protocols [Internet]. Humana Press; 2010 [cited 2014 Sep. 5]. p. 103-9. (Methods in Molecular Biology). Available from: http://link.springer.com/protocol/10.1007/978-1-60761-652-8_7
13. Binkowski B F, Richmond K E, Kaysen J, Sussman M R, Belshaw P J. Correcting errors in synthetic DNA through consensus shuffling. Nucleic Acids Res. 2005 Jan. 1; 33(6):e55-e55.
14. Crameri A, Raillard S A, Bermudez E, Stemmer W P. DNA shuffling of a family of genes from diverse species accelerates directed evolution. Nature. 1998 Jan. 15; 391(6664):288-91.
15. Stemmer W P. DNA shuffling by random fragmentation and reassembly: in vitro recombination for molecular evolution. Proc Natl Acad Sci. 1994 Oct. 25; 91(22):10747-51.
16. Stemmer W P C. Rapid evolution of a protein in vitro by DNA shuffling. Nature. 1994 Aug. 4; 370(6488):389-91.
17. Barettino D, Feigenbutz M, Valcarcel R, Stunnenberg H G. Improved method for PCR-mediated site-directed mutagenesis. Nucleic Acids Res. 1994 Feb. 11; 22(3):541-2.
18. Steffens D L, Williams J G K. Efficient Site-Directed Saturation Mutagenesis Using Degenerate Oligonucleotides. J Biomol Tech JBT. 2007 July; 18(3):147-9.
19. Braman J, Papworth C, Greener A. Site-Directed Mutagenesis Using Double-Stranded Plasmid DNA Templates. In: Trower M, editor. In Vitro Mutagenesis Protocols [Internet]. Humana Press; 1996 [cited 2016 Feb. 16]. p. 31-44. (Methods In Molecular Medicine™). Available from: http://dx.doi.org/10.1385/0-89603-332-5%3A31
20. Currin A, Swainston N, Day P J, Kell D B. SpeedyGenes: an improved gene synthesis method for the efficient production of error-corrected, synthetic protein libraries for directed evolution. Protein Eng Des Sel. 2014 Sep. 1; 27(9):273-80.
21. Reetz M T, Kahakeaw D, Lohmer R. Addressing the Numbers Problem in Directed Evolution. ChemBioChem. 2008 Jul. 21; 9(11):1797-804.
22. Tang L, Gao H, Zhu X, Wang X, Zhou M, Jiang R. Construction of “small-intelligent” focused mutagenesis libraries using well-designed combinatorial degenerate primers. BioTechniques. 2012 March; 52(3):149-58.
23. Wang X, Zheng K, Zheng H, Nie H, Yang Z, Tang L. D C-Analyzer-facilitated combinatorial strategy for rapid directed evolution of functional enzymes with multiple mutagenesis sites. J Biotechnol. 2014 Dec. 20; 192, Part A:102-7.
24. Wang X, Lin H, Zheng Y, Feng J, Yang Z, Tang L. MDC-Analyzer-facilitated combinatorial strategy for improving the activity and stability of halohydrin dehalogenase from Agrobacterium radiobacter AD1. J Biotechnol. 2015 Jul. 20; 206:1-7.
25. Bi Y, Qiao X, Hua Z, Zhang L, Liu X, Li L, et al. An asymmetric PCR-based, reliable and rapid single-tube native DNA engineering strategy. BMC Biotechnol. 2012 Jul. 6; 12(1):39.
26. Warrens A N, Jones M D, Lechler R I. Splicing by overlap extension by PCR using asymmetric amplification: an improved technique for the generation of hybrid proteins of immunological interest. Gene. 1997 Feb. 20; 186(1):29-35.
27. Perrin S, Gilliland G. Site-specific mutagenesis using asymmetric polymerase chain reaction and a single mutant primer. Nucleic Acids Res. 1990 Dec. 25; 18(24):7433-8.
28. Wang B-L, Jiao Y-L, Li X-X, Zheng F, Liang H, Sun Z-Y, et al. A universal method for directional cloning of PCR products based on asymmetric PCR. Biotechnol Appl Biochem. 2009 Jan. 1; 52(1):41-4.
29. Xiao Y-H, Yin M-H, Hou L, Luo M, Pei Y. Asymmetric overlap extension PCR method bypassing intermediate purification and the amplification of wild-type template in site-directed mutagenesis. Biotechnol Lett. 2007 Jun. 1; 29(6):925-30.
30. Rowles I, Malone K J, Etchells L L, Willies S C, Turner N J. Directed Evolution of the Enzyme Monoamine Oxidase (MAO-N): Highly Efficient Chemo-enzymatic Deracemisation of the Alkaloid (±)-Crispine A. ChemCatChem. 2012; 4(9):1259-61.
31. Cunningham B C, Wells J A. High-resolution epitope mapping of hGH-receptor interactions by alanine-scanning mutagenesis. Science. 1989 Jun. 2; 244(4908):1081-5.
32. Lefèvre F, Rémy M-H, Masson J-M. Alanine-stretch scanning mutagenesis: a simple and efficient method to probe protein structure and function. Nucleic Acids Res. 1997 Jan. 1; 25(2):447-8.
33. Reetz M T, Wang L-W, Bocola M (2006) Directed Evolution of Enantioselective Enzymes: Iterative Cycles of CASTing for Probing Protein-Sequence Space. Angew Chem Int Ed 45(8):1236-1241.
34. Reetz M T, et al. (2006) Expanding the Substrate Scope of Enzymes: Combining Mutations Obtained by CASTing. Chem—Eur J 12(23):6031-6038.
35. Alexeeva M, Enright A, Dawson M J, Mahmoudian M, Turner N J (2002) Deracemization of α-Methylbenzylamine Using an Enzyme Obtained by In Vitro Evolution. Angew Chem Int Ed 41(17):3177-3180.
36. Atkin K E, et al. (2008) The Structure of Monoamine Oxidase from Aspergillus niger Provides a Molecular Context for Improvements in Activity Obtained by Directed Evolution. J Mol Biol 384(5):1218-1231.
37. Dunsmore C J, Carr R, Fleming T, Turner N J (2006) A Chemo-Enzymatic Route to Enantiomerically Pure Cyclic Tertiary Amines. J Am Chem Soc 128(7):2224-2225.
40. Bradley L H, Thumfort P P, Hecht M H (2006) De novo proteins from binary-patterned combinatorial libraries. Protein Design, Methods in Molecular Biology., eds Guerois R, Paz M L (Humana Press), pp 53-69 Reetz M T, et al. (2006) Expanding the Substrate Scope of Enzymes: Combining Mutations Obtained by CASTing. Chem—Eur J 12(23):6031-6038.
41. Currin A, Swainston N, Day P J, Kell D B (2015) Synthetic biology for the directed evolution of protein biocatalysts: navigating sequence space intelligently. Chem Soc Rev 44(5):1172-1239.
42. Alexeeva M, Carr R, Turner N J (2003) Directed evolution of enzymes: new biocatalysts for asymmetric synthesis. Org Biomol Chem 1(23):4133-4137.
43. Morley K L, Kazlauskas R J (2005) Improving enzyme properties: when are closer mutations better? Trends Biotechnol 23(5):231-237.
44. Jiménez-Osés G, et al. (2014) The role of distant mutations and allosteric regulation on LovD active site dynamics. Nat Chem Biol 10(6):431-436.
45. Romero-Rivera A, Garcia-Borras M, Osuna S (2016) Computational tools for the evaluation of laboratory-engineered biocatalysts. Chem Commun 53(2):284-297.
46. Knight C G, et al. (2009) Array-based evolution of DNA aptamers allows modelling of an explicit sequence-fitness landscape. Nucleic Acids Res 37(1):e6.
47. Fox R J, Huisman G W (2008) Enzyme optimization: moving from blind evolution to statistical exploration of sequence-function space. Trends Biotechnol 26(3):132-138
48. Fox R J, et al. (2007) Improving catalytic function by ProSAR-driven enzyme evolution. Nat Biotechnol 25(3):338-344.
49. Berland M, Offmann B, André I, Remaud-Simeon M, Charton P (2014) A web-based tool for rational screening of mutants libraries using ProSAR. Protein Eng Des Sel 27(10): 375-381
50. Fowler D M, Stephany J J, Fields S (2014) Measuring the activity of protein variants on a large scale using deep mutational scanning. Nat Protoc 9(9):2267-2284.
51. Starita L M, Fields S (2015) Deep Mutational Scanning: A Highly Parallel Method to Measure the Effects of Mutation on Protein Function. Cold Spring Harb Protoc 2015(8):pdb.top077503
52. Kell D B (2012) Scientific discovery as a combinatorial optimisation problem: How best to navigate the landscape of possible experiments? Bioessays 34(3):236-244.
54. Miyamoto T, Razavi S, DeRose R, Inoue T (2013) Synthesizing Biomolecule-Based Boolean Logic Gates. ACS Synth Biol 2(2):72-82
55. Li G, et al. (2017) Simultaneous engineering of an enzyme's entrance tunnel and active site: the case of monoamine oxidase MAO-N. Chem Sci 8(5):4093-4099.
56. Bailey K R, Ellis A J, Reiss R, Snape T J, Turner N J (2007) A template-based mnemonic for monoamine oxidase (MAO-N) catalyzed reactions and its application to the chemo-enzymatic deracemisation of the alkaloid (±)-crispine A. Chem Commun (35):3640.
57. Ghislieri D, Houghton D, Green A P, Willies S C, Turner N J (2013) Monoamine Oxidase (MAO-N) Catalyzed Deracemization of Tetrahydro-β-carbolines: Substrate Dependent Switch in Enantioselectivity. ACS Catal 3(12):2869-2872.
58. O'Reilly E, et al. (2014) A Regio- and Stereoselective ω-Transaminase/Monoamine Oxidase Cascade for the Synthesis of Chiral 2,5-Disubstituted Pyrrolidines. Angew Chem Int Ed 53(9):2447-2450.

1.-52. (canceled)

53. A method of making a variant library of a nucleic acid molecule of interest, wherein the library comprises a population of variant nucleic acid molecules, wherein each variant comprises an alternative (OR-type) mutation in at least one target region, the method comprising:
I) incubating i) a nucleic acid molecule of interest comprising two strands; ii) a limited concentration of two or more mutagenic primers which each hybridise to a first strand of the nucleic acid molecule of interest; and iii) an excess concentration of a first primer which hybridises to a second strand of the nucleic acid molecule of interest, to obtain a first incubation;
II) maintaining the first incubation under suitable conditions for X number of rounds of a first amplification reaction, wherein X is n+y where n is the number of amplification rounds required to deplete the one or more mutagenic primers, and y is 2 or more; to generate two or more single stranded first amplification reaction products of a first target region wherein each of the two or more single stranded first amplification reaction products comprises an alternate (OR-type) mutation in a target region and is capable of hybridising to the second strand of the nucleic acid molecule of interest;
III) incubating i) the two or more said single stranded first amplification reaction products of the first target region which hybridise to the second strand of the nucleic acid molecule of interest; and ii) a second primer which hybridises to the first strand of the nucleic acid molecule of interest to obtain a second incubation to provide two or more double stranded second amplification reaction products; and
IV) maintaining the second incubation under suitable conditions for sufficient rounds of a second amplification reaction to generate the two or more double stranded second amplification reaction products wherein each double stranded second amplification reaction product comprises an alternate (OR-type) mutation.

54. The method of claim 53 wherein the nucleic acid molecule of interest is notionally or physically divided into at least 2, 3, 4, 5, 6, 7, or 8 or more target regions.

55. The method of claim 53 wherein each variant of the library further comprises an alternate (OR-type) mutation in a second or further target region, the method further comprising:
in step I) incubating in the first incubation i) a limited concentration of two or more second or further mutagenic primers which each hybridise to a first strand of the nucleic acid molecule of interest for amplification of a second or further target region; and ii) an excess concentration of a first primer which hybridises to the second strand of the nucleic acid molecule of interest;
in step II) maintaining the first incubation under suitable conditions for X number of rounds of a third or further amplification reaction, wherein X is n+y where n is the number of amplification rounds required to deplete the one or more second or further mutagenic primers, and y is 2 or more; to generate two or more single stranded third or further amplification reaction products of a second or further target region, each two or more single stranded third or further amplification reaction products each comprising an alternate (OR-type) mutation in the first target region and an alternate (OR-type) mutation in the second or further target region;
in step III) incubating in the second incubation i) the two or more said single stranded third or further amplification reaction products of the second or further target region which hybridise to the second strand of the nucleic acid molecule of interest; and ii) a second primer which hybridises to the first strand of the nucleic acid molecule of interest to provide two or more double stranded fourth or further amplification reaction products of the second or further target region; and
in step IV), maintaining the second incubation under suitable conditions for sufficient rounds of a fourth or further amplification reaction to generate the two or more double stranded fourth or further amplification reaction products of the second or further target region wherein each double stranded fourth or further amplification reaction product comprises an alternate (OR-type) mutation in the second or further target region.

56. The method of claim 53 wherein the first amplification reaction of step II) amplifies said two or more target regions simultaneously.

57. The method of claim 53 wherein amplification of a target region with each of said two or more mutagenic primers is performed separately, sequentially or simultaneously.

58. The method of claim 55 wherein the mutation or mutations introduced into the second or further target regions are not shared by another variant.

59. The method of claim 54 wherein the first and/or second primer is common to two or more target regions, or wherein different first and/or second primers are provided for any two or more target regions.

60. The method of claim 53 wherein at least one mutagenic primer is provided in a concentration such that it becomes depleted in n number of PCR rounds in an amplification reaction, wherein n is fewer than a total number of PCR cycles in the amplification reaction.

61. The method of claim 53 wherein the first primer is provided in a greater concentration than the concentration of at least one mutagenic primer.

62. The method of claim 53 wherein the first and/or second primer is non-mutagenic, such that it has a sequence which is sufficiently complementary to a primer binding site of the nucleic molecule of interest that it does not introduce any mutation into an amplification product.

63. The method of claim 53 wherein the mutagenic primers are designed to i) introduce a walking mutation in a variant nucleic acid molecule library or a mutation at alternate nucleotides and/or ii) introduce the same type of mutation (addition, substitution or deletions) at each mutation site.

64. The method of claim 53 wherein two or more different mutagenic primers target the same (first, second or further) target region or two or more different (first, second and further) target regions.

65. The method of claim 53 wherein the two or more single stranded first amplification products and the second primer are provided in approximately equal concentrations to generate a double stranded second amplification reaction product.

66. The method of claim 53 wherein the two or more single stranded amplification products are isolated from the first amplification reaction, for use in the second amplification reaction.

67. The method of claim 53 comprising providing two or more second primers for use in the second amplification reaction in the second incubation, optionally together with a polymerase and nucleotides.

68. The method of claim 67 wherein the two or more second primers are mutagenic.

69. The method of claim 53 further comprising one or more of i) cloning the two or more amplification products into a vector; ii) transforming a host cell with said vector to produce a library of said nucleic acid molecule of interest; iii) selecting a desired clone from the library; iv) isolating and purifying the variant nucleic acid molecule from the clone; v) cloning the nucleic acid molecule into an expression vector; and vi) transforming a host to allow expression of the expression vector.

70. The method of claim 53 comprising preparing a protein library recombinantly by expression to obtain expressed protein, and optionally purification of the expressed protein, in a host cell or a cell-free system.

71. A variant nucleic acid library of a nucleic acid molecule of interest, wherein each variant comprises an alternate (OR-type) mutation in a first target region, to other variants in the population.

72. The variant nucleic acid library of claim 71 wherein each variant comprises an alternate (OR-type) mutation in a second or further target region, to other variants in the population.

73. The variant nucleic acid library of claim 71 wherein each variant is a nucleic acid molecule comprising an alternate (OR-type) mutation at two or more target regions simultaneously.

74. The variant nucleic acid library of claim 71 wherein each nucleic acid variant comprises a different number of alternate (OR-type) mutations in any one target region.

75. The variant nucleic acid library of claim 71 wherein each variant is a nucleic acid molecule comprising an alternate (OR-type) mutation in a single (first, second, third or further) target region.

76. The variant nucleic acid library of claim 71, wherein each variant comprises an alternate (OR-type) mutation in one or more target regions and a non-alternate (not OR-type) mutation in one or more other target regions.

77. The variant nucleic acid library of claim 71 which comprises a physical library that is smaller than a theoretical library.

78. A population of variant host cells, each variant comprising a variant nucleic acid molecule of a library, wherein each variant nucleic acid molecule comprises an alternative (OR-type) mutation in a first target region to other members of the population.
